SEQUENCE LISTING (9 sequences)

SEQ ID NO: 1 - protein, 288 aa, Homo sapiens
  REGION 1..288: note = human PDCD1, UniProt accession number Q15116
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 287 aa, Homo sapiens
  REGION 1..287: note = human LAIR-1a, UniProtKB - Q6GTX8 (LAIR1_HUMAN)
MSPHPTALLGLVLCLAQTIHTQEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 3 - protein, 144 aa, Homo sapiens
  REGION 1..144: note = extracellular domain for human LAIR-1
QEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLY

SEQ ID NO: 4 - protein, 152 aa, Homo sapiens
  REGION 1..152: note = human LAIR-2a, UniProtKB - Q6ISS4 (LAIR2_HUMAN)
MSPHLTALLGLVLCLAQTIHTQEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAP

SEQ ID NO: 5 - protein, 131 aa, Homo sapiens
  REGION 1..131: note = Leukocyte-associated immunoglobulin-like receptor 2 domain for human LAIR-2
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAP

SEQ ID NO: 6 - protein, 376 aa, synthetic construct
  REGION 1..376: note = Synthetic: human LAIR2-hIg fusion protein (hIgG1) (hLAIR2.hG1)
MEWSWVFLFFLSVTTGVHSQEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 357 aa, synthetic construct
  REGION 1..357: note = Synthetic: SEQ ID NO:6 without the signal sequence
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 382 aa, synthetic construct
  REGION 1..382: note = Synthetic: human LAIR2.mIg fusion protein (mIgG2a)
MEWSWVFLFFLSVTTGVHSQEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 9 - protein, 363 aa, synthetic construct
  REGION 1..363: note = Synthetic: SEQ ID NO:8 without the signal sequence
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

(19) United States
(12) Patent Application Publication
(10) Pub. No.: US 2026/0193349 A1
(43) Pub. Date: Jul. 9, 2026

(54) IMMUNE CHECKPOINT INHIBITOR AND EXTRACELLULAR MATRIX COMPONENT BINDER COMBINATION THERAPY AND METHODS OF USE THEREOF

(71) Applicant: NEXTCURE, INC., Beltsville, MD (US)

(72) Inventors: Han MYINT, Beltsville, MD (US); Solomon LANGERMANN, Beltsville, MD (US)

(73) Assignee: NEXTCURE, INC., Beltsville, MD (US)

(21) Appl. No.: 19/128,152

(22) PCT Filed: Nov. 8, 2023
(86) PCT No.: PCT/US2023/079069

§ 371 Date: May 7, 2025

Related U.S. Application Data
(60) Provisional application No. 63/382,845, filed on Nov. 8, 2022.

(51) Int. Cl. C07K 16/28; A61K 38/00; A61K 39/00; A61P 35/00; C07K 14/705

(52) U.S. Cl. CPC C07K 16/2818; A61P 35/00; C07K 14/70503; A61K 38/00; A61K 2039/505; C07K 2319/30

(57) ABSTRACT

A clinical combination therapy including one or more pharmaceutical compositions to treat various cancers. One or more pharmaceutical compositions include at least a first composition of an immune checkpoint inhibitor and a second composition that interacts with components of the extracellular matrix of a tumor microenvironment. A first composition may comprise, as an example, Pembrolizumab, while a second composition may comprise a LAIR-2 Fc fusion protein such as NC410. The compositions are subject to dosing and timing regimens.

### TECHNICAL FIELD

This disclosure generally relates to the field of combination therapies for the treatment of cancers.

### BACKGROUND

The importance of intact immune surveillance function in controlling outgrowth of neoplastic transformations has been known for decades (Disis, M. L., “Immune regulation of cancer”, J Clin Oncol., 28:4531-8 (2010)). Accumulating evidence shows a correlation between tumor-infiltrating lymphocytes in cancer tissue and favorable prognosis in various malignancies. In particular, the presence of CD8+ T-cells and the ratio of CD8+ effector T-cells/FoxP3+ regulatory T-cells (T-regs) correlates with improved prognosis and long-term survival in solid malignancies, such as ovarian, colorectal, and pancreatic cancer; hepatocellular carcinoma; malignant melanoma; and renal cell carcinoma. Tumor-infiltrating lymphocytes can be expanded ex vivo and reinfused, inducing durable objective tumor responses in cancers such as melanoma (Dudley, M. E., et al., “Adoptive cell transfer therapy following non-myeloablative but lymphodepleting chemotherapy for the treatment of patients with refractory metastatic melanoma”, J Clin Oncol., 23:2346-57 (2005); Hunder, N. N., et al., “Treatment of metastatic melanoma with autologous CD4+ T cells against NY-ESO-1”, N Engl J Med., 358:2698-703 (2008)).

Therapeutic studies in mouse models have shown that administration of antibodies blocking PD-1/PD-L1 interaction enhances infiltration of tumor-specific CD8+ T cells and ultimately leads to tumor rejection, either as a monotherapy or in combination with other treatment modalities (Strome, S. E, et al., “B7-H1 blockade augments adoptive T-cell immunotherapy for squamous cell carcinoma”, Cancer Res., 63:6501-5 (2003); Blank, C., et al., “PD-L1/B7H-1 inhibits the effector phase of tumor rejection by T cell receptor (TCR) transgenic CD8+ T cells”, Cancer Res., 64:1140-5 (2004); Hirano, F., et al., “Blockade of B7-H1 and PD-1 by monoclonal antibodies potentiates cancer therapeutic immunity”, Cancer Res., 65:1089-96 (2005); Curran, M. A., et al., “PD-1 and CTLA-4 combination blockade expands infiltrating T cells and reduces regulatory T and myeloid cells within B16 melanoma tumors”, Proc Natl Acad Sci U.S.A., 107:4275-80 (2010); Pilon-Thomas, S., et al., “Blockade of programmed death ligand 1 enhances the therapeutic efficacy of combination immunotherapy against melanoma”, J Immunol., 184:3442-9 (2010); Weber, J., “Immune checkpoint proteins: a new therapeutic paradigm for cancer—preclinical background: CTLA-4 and PD-1 blockade”, Semin Oncol., 37:430-9 (2010); Spranger, S., et al., “Mechanism of tumor rejection with doublets of CTLA-4, PD-1/PD-L1, or IDO blockade involves restored IL-2 production and proliferation of CD8(+) T cells directly within the tumor microenvironment”, J Immunother Cancer., 2:3 (2014)). Anti-mouse PD-1 or anti-mouse PD-L1 antibodies have demonstrated antitumor responses in models of squamous cell carcinoma, pancreatic carcinoma, melanoma, acute myeloid leukemia and colorectal carcinoma (Strome, S. E, et al., “B7-H1 blockade augments adoptive T-cell immunotherapy for squamous cell carcinoma”, Cancer Res., 63:6501-5 (2003); Nomi, 2007; Zhang, 2009; Curran, M. A., et al., “PD-1 and CTLA-4 combination blockade expands infiltrating T cells and reduces regulatory T and myeloid cells within B16 melanoma tumors”, Proc Natl Acad Sci U.S.A., 107:4275-80 (2010); Pilon-Thomas, S., et al., “Blockade of programmed death ligand 1 enhances the therapeutic efficacy of combination immunotherapy against melanoma”, J Immunol., 184:3442-9 (2010)). In such studies, tumor infiltration by CD8+ T cells and increased IFN-γ, granzyme B and perforin expression were observed, indicating that the mechanism underlying the antitumor activity of PD-1 checkpoint inhibition involved local infiltration and activation of effector T cell function in vivo (Curran, M. A., et al., “PD-1 and CTLA-4 combination blockade expands infiltrating T cells and reduces regulatory T and myeloid cells within B16 melanoma tumors”, Proc Natl Acad Sci U.S.A., 107:4275-80 (2010)). Experiments have confirmed the in vivo efficacy of anti-mouse PD-1 antibody as a monotherapy, as well as in combination with chemotherapy, in syngeneic mouse tumor models. However, immune checkpoint inhibitors (ICI) such as anti-PD-1 antibodies can be limited as a result of components within the extracellular matrix (ECM) of a tumor microenvironment (TME) that may bind to the ICI.

There remains a need in the art to improve functions of immune checkpoint inhibitors in tumor treatment, reduction, and killing.

### INCORPORATION BY REFERENCE

Any foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

Citation or identification of any document in this application is not an admission that such document is available as prior art to the present invention.

### SUMMARY

It is to be understood that this summary is not an extensive overview of the disclosure. This summary is exemplary and not restrictive, and it is intended to neither identify key or critical elements of the disclosure nor delineate the scope thereof. The sole purpose of this summary is to explain and exemplify certain concepts of the disclosure as an introduction to the following complete and extensive detailed description.

The present disclosure is related to a combination therapy comprising at least two pharmaceutical compositions. The combination therapy substantially treats or reduces symptoms of diseases including but not limited to cancer. Cancer includes but is not limited to colorectal cancer, gastric cancer, gastroesophageal junction cancer, esophageal cancer, endometrial cancer, or head and neck cancer.

A first pharmaceutical composition includes an immune checkpoint inhibitor (ICI) such as an ICI that targets the Programmed Cell Death Protein 1 (PD-1) pathway. Such an ICI includes but is not limited to Pembrolizumab. The first pharmaceutical composition can be administered on the first day of a repeating 42-day cycle at an approximate dosage of about 400 mg. Alternatively, pembrolizumab may be administered at a dose of 200 mg every 21 days.

A second pharmaceutical composition includes a protein configured to bind to one or more components, such as collagen or C1q, of an extracellular matrix (ECM) of a tumor microenvironment (TME). Such a protein includes but is not limited to a LAIR-2 protein, a LAIR-2 functional fragment, a LAIR-2 variant, and a LAIR-2 fusion protein (e.g., NC410). A LAIR-2 protein or functional fragment or variant can comprise at least 80%, 90%, 95%, or 100% sequence identity to SEQ ID NO: 5. The second pharmaceutical composition can be administered on days 1, 15, and 29 of a repeating 42-day cycle. Potential doses of the second pharmaceutical composition include about 15 mg, about 30 mg, about 60 mg, about 100 mg, about 200 mg, about 250 mg, about 300 mg, about 350 mg, and about 400 mg. The second pharmaceutical composition can be administered on a variable schedule for a variable length of time as can be determined by considering usual factors and routine experimentation known to one of skill in the art. In one embodiment of the present invention, the composition is administered on days 1, 15, and 29 of a repeating 42-day cycle at about 15 mg, about 30 mg, about 60 mg, about 100 mg, or about 200 mg. In another embodiment, the second pharmaceutical composition can be administered on a weekly basis of a repeating 42-day cycle at about 100 mg.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 displays the Müllerian inhibiting substance type II receptor (MISIIR) transgenic spontaneous model of ovarian cancer, LAIR-1 is expressed on CD11c+CD11b+ suppressive dendritic cells (DCs) at primary and metastatic sites of disease. LAIR-1 is not expressed on CD103+stimulatory DCs.

FIG. 2 displays NC410 binding to tumor-associated ligands (collagen and C1q) to block LAIR-1 inhibition and promote adaptive (T cells) and innate (dendritic cells) immune responses, as well as to activate macrophages, ultimately resulting in tumor cell killing.

FIG. 3 displays a dose escalation study design according to the present disclosure.

FIG. 4 displays an exemplary dosing and timing regimen according to the present disclosure.

FIG. 5 displays a dose-finding rule matrix according to the present disclosure.

FIG. 6 displays the analysis of five representative samples of stomach adenocarcinoma (STAD) analyzed by H&E staining, for LAIR-2-Fc (NC410) binding and by immuno staining of LAIR-1, CD45, CD3 and CD163 positive cells.

FIG. 7 displays dose finding rules according to a Simon 2 Stage Design of the present disclosure.

FIG. 8 displays collagen staining by trichome staining (Left) and LAIR-1 staining in immune cells (Right) by IHC in multiple tumor types.

FIG. 9 displays a line graph showing that NC410 in combination with anti-PD-L1 results in synergistic and reproducible tumor killing in murine models. NC410 at 200 ug Q4D for 5 doses±PD-L1 at 100 ug Q7D for 2 doses.

### DETAILED DESCRIPTION

The present disclosure can be understood more readily by reference to the following detailed description, examples, drawings, and claims, and their previous and following description. However, before the present compositions and/or methods are disclosed and described, it is to be understood that this disclosure is not limited to the specific compositions and/or methods disclosed unless otherwise specified, as such can, of course, vary. It is also to be understood that the terminology used herein is for the purpose of describing particular aspects only and is not intended to be limiting.

### I. Definitions

Unless defined otherwise, all technical and scientific terms used herein have the same meaning as commonly understood by one of ordinary skill in the art to which this disclosure belongs. Although any compositions, methods, and materials similar or equivalent to those described herein can be used in the practice or testing of the present invention. All publications mentioned are incorporated herein by reference in their entirety.

Unless defined otherwise, all composition percentage values used herein are given in terms of weight percentage.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “administration” as it applies to a human, primate, mammal, mammalian subject, animal, veterinary subject, placebo subject, research subject, experimental subject, cell, tissue, organ, or biological fluid, refers to contact of an exogenous ligand, reagent, placebo, small molecule, pharmaceutical agent, therapeutic agent, diagnostic agent, or composition to the subject, cell, tissue, organ, or biological fluid, and the like. “Administration” can refer to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration” also encompasses in vitro and ex vivo treatments, e.g., of a cell, by a reagent, diagnostic, binding composition, or by another cell.

An “agonist,” as it relates to a ligand and receptor, comprises a molecule, combination of molecules, a complex, or a combination of reagents, that stimulates the receptor. For example, an agonist of granulocyte-macrophage colony stimulating factor (GM-CSF) can encompass GM-CSF, a mutein or derivative of GM-CSF, a peptide mimetic of GM-CSF, a small molecule that mimics the biological function of GM-CSF, or an antibody that stimulates GM-CSF receptor.

As used herein, an “analog” or “derivative” with reference to a peptide, polypeptide or protein refers to another peptide, polypeptide or protein that possesses a similar or identical function as the original peptide, polypeptide, or protein, but does not necessarily comprise a similar or identical amino acid sequence or structure of the original peptide, polypeptide, or protein. An analog preferably satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the original amino acid sequence (b) a proteinaceous agent encoded by a nucleotide sequence that hybridizes under stringent conditions to a nucleotide sequence encoding the original amino acid sequence; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding the original amino acid sequence.

As used herein, the term “antibody” is intended to denote an immunoglobulin molecule that possesses a “variable region” antigen recognition site. The term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies). In particular, such antibodies include immunoglobulin molecules of any type (e.g., IgG, IgE, IgM, IgD, IgA and IgY), class (e.g., IgG1, IgG2, IgG3, IgG4, IgA1 and IgA2) or subclass.

As used herein, “antigen presenting cells” (APCs) are cells of the immune system used for presenting antigen to T cells. APCs include dendritic cells, monocytes, macrophages, marginal zone Kupffer cells, microglia, Langerhans cells, T cells, and B cells. Dendritic cells occur in at least two lineages. The first lineage encompasses pre-DC1, myeloid DC1, and mature DC1. The second lineage encompasses CD34⁺CD45RA⁻ early progenitor multipotent cells, CD34⁺CD45RA⁺ cells, CD34⁺CD45RA⁺CD4⁺ IL-3Rα⁺ pro-DC2 cells, CD4⁺CD11c⁻ plasmacytoid pre-DC2 cells, lymphoid human DC2 plasmacytoid-derived DC2s, and mature DC2s.

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site and exhibit an ability to immunospecifically bind antigen. Such fragments include Fab′, F(ab′)₂, Fv, single chain (ScFv), and mutants thereof, naturally occurring variants, and fusion proteins including the antibody's “variable region” antigen recognition site and a heterologous protein (e.g., a toxin, an antigen recognition site for a different antigen, an enzyme, a receptor, or receptor ligand, etc.).

As used herein, “attenuated gene” encompasses a gene that mediates toxicity, pathology, or virulence, to a host, growth within the host, or survival within the host, where the gene is mutated in a way that mitigates, reduces, or eliminates the toxicity, pathology, or virulence. The reduction or elimination can be assessed by comparing the virulence or toxicity mediated by the mutated gene with that mediated by the non-mutated (or parent) gene. “Mutated gene” encompasses deletions, point mutations, and frameshift mutations in regulatory regions of the gene, coding regions of the gene, non-coding regions of the gene, or any combination thereof.

As used herein, the term “cancer” refers to a neoplasm or tumor resulting from abnormal uncontrolled growth of cells. As used herein, cancer explicitly includes, leukemias and lymphomas. The term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or web-like matrices in a three-dimensional basement membrane or extracellular matrix preparation. Non-cancer cells do not form colonies in soft agar and form distinct sphere-like structures in three-dimensional basement membrane or extracellular matrix preparations.

As used herein, a “chimeric antibody” is a molecule in which different portions of the antibody are derived from different immunoglobulin molecules such as antibodies having a variable region derived from a non-human antibody and a human immunoglobulin constant region.

As used herein, the term “chimeric receptor” is defined as a cell-surface receptor comprising an extracellular ligand binding domain, a transmembrane domain and a cytoplasmic co-stimulatory signaling domain in a combination that is not naturally found together on a single protein. This particularly includes receptors wherein the extracellular domain and the cytoplasmic domain are not naturally found together on a single receptor protein. Further, the chimeric receptor is different from the TCR expressed in the native T cell lymphocyte.

As used herein, the “co-stimulatory” signals encompass positive co-stimulatory signals (e.g., signals that result in enhancing an activity) and negative co-stimulatory signals (e.g., signals that result in inhibiting an activity).

Throughout this specification, unless the context requires otherwise, the words “comprise”, “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “derivative” refers to an antibody or antigen-binding fragment thereof that immunospecifically binds to the same target of a parent or reference antibody, but which differs in amino acid sequence from the parent or reference antibody or antigen binding fragment thereof by including one, two, three, four, five or more amino acid substitutions, additions, deletions, or modifications relative to the parent or reference antibody or antigen binding fragment thereof. In some embodiments such derivatives will have substantially the same immunospecificity and/or characteristics, or the same immunospecificity and characteristics as the parent or reference antibody or antigen binding fragment thereof. The amino acid substitutions or additions of such derivatives can include naturally occurring (i.e., DNA-encoded) or non-naturally occurring amino acid residues. The term “derivative” encompasses, for example, chimeric or humanized variants, as well as variants having altered CH1, hinge, CH2, CH3 or CH4 regions, so as to form, for example antibodies, etc., having variant Fc regions that exhibit enhanced or impaired effector or binding characteristics.

As used herein, “effective amount” encompasses, without limitation, an amount (e.g., of a protein, polypeptide, fragments thereof, etc.) that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition.

Reference throughout this specification to “embodiment,” “one embodiment,” “an embodiment,” “a particular embodiment,” “a related embodiment,” “a certain embodiment,” “an additional embodiment,” or “a further embodiment” or combinations thereof means that a particular feature, structure, or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “endogenous concentration” refers to the level at which a molecule is natively expressed (i.e., in the absence of expression vectors or recombinant promoters) by a cell (which cell can be a normal cell, a cancer cell or an infected cell).

As used herein, “epitope” refers to an antigenic determinant capable of specific binding to an antibody. Epitopes usually consist of chemically active surface groupings of molecules such as amino acids or sugar side chains and usually have specific three-dimensional structural characteristics, as well as specific charge characteristics. Conformational and nonconformational epitopes are distinguished in that the binding to the former but not the latter is lost in the presence of denaturing solvents.

As used herein, “extracellular fluid” encompasses serum, plasma, blood, interstitial fluid, cerebrospinal fluid, secreted fluids, lymph, bile, sweat, fecal matter, and urine. An “extracellular fluid” can comprise a colloid or a suspension, such as whole blood or coagulated blood.”

As used herein, “fragments” in the context of polypeptides include a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a larger polypeptide.

As used herein, the term “humanized antibody” refers to an immunoglobulin including a human framework region and one or more CDR's from a non-human (usually a mouse or rat) immunoglobulin. The non-human immunoglobulin providing the CDR's is called the “donor” and the human immunoglobulin providing the framework is called the “acceptor.” Constant regions need not be present, but if they are, they should be substantially identical to human immunoglobulin constant regions, i.e., at least about 85-99%, or about 95% or more identical. Hence, all parts of a humanized immunoglobulin, except possibly the CDR's, are substantially identical to corresponding parts of natural human immunoglobulin sequences. A humanized antibody is an antibody including a humanized light chain and a humanized heavy chain immunoglobulin. For example, a humanized antibody would not encompass a typical chimeric antibody, because, e.g., the entire variable region of a chimeric antibody is non-human.

As used herein, an “immune cell” refers to any cell from the hemopoietic origin including, but not limited to, T cells, B cells, monocytes, dendritic cells, and macrophages.

As used herein, “immune checkpoints” refer to inhibitory pathways of the immune system that are responsible for maintaining self-tolerance and modulating the duration and amplitude of physiological immune responses in peripheral tissues in order to minimize collateral tissue damage. Immune checkpoints are regulated by immune checkpoint proteins.

As used herein, an “immune checkpoint protein” is a protein, typically a receptor (e.g., CTLA4 or PD-1) or a ligand (e.g., PD-L1) that regulates or modulates the extent of an immune response. The immune checkpoint proteins can be inhibitory or stimulatory. In particular, the immune checkpoint proteins are inhibitory to the activation of the immune response. Thus, inhibition of an inhibitory immune checkpoint protein acts to stimulate or activate an immune response, such as T cell activation and proliferation.

As used herein, an “immune checkpoint inhibitor” or “immune checkpoint inhibiting agent,” or “immune checkpoint blocking agent” refers to an agent that binds an inhibitory immune checkpoint protein and blocks its activity. The inhibition can be competitive or non-competitive inhibition that can be steric or allosteric. In cases where an immune checkpoint protein is an immune stimulating protein, an immune checkpoint inhibitor acts to promote the activity of the immune stimulating protein, such as by binding and activating the stimulatory immune checkpoint protein or by inhibiting by interfering with, such as by binding or deactivating, inhibitors of the stimulatory immune checkpoint protein. An example of an immune checkpoint inhibitor is an anti-immune checkpoint protein antibody.

A “target” of an-immune checkpoint inhibitor is the immune checkpoint protein to which the immune checkpoint inhibitor or immune checkpoint inhibiting agent binds to block activity. Typically, the immune checkpoint inhibitor specifically binds to the target. For example, the target of the exemplary anti-CTLA4 antibody designated Ipilimumab is CTLA4.

An “immunogenic agent” or “immunogen” is capable of inducing an immunological response against itself on administration to a mammal, optionally in conjunction with an adjuvant.

As used herein, the terms “immunologic,” “immunological” or “immune” response is the development of a beneficial humoral (antibody mediated) and/or a cellular (mediated by antigen-specific T cells or their secretion products) response directed against a peptide in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

As used herein, a molecule is said to be able to “immunospecifically bind” a second molecule if such binding exhibits the specificity and affinity of an antibody to its cognate antigen. Antibodies are said to be capable of immunospecifically binding to a target region or conformation (“epitope”) of an antigen if such binding involves the antigen recognition site of the immunoglobulin molecule. An antibody that immunospecifically binds to a particular antigen may bind to other antigens with lower affinity if the other antigen has some sequence or conformational similarity that is recognized by the antigen recognition site as determined by, e.g., immunoassays, BIACORE® assays, or other assays known in the art, but would not bind to a totally unrelated antigen. In some embodiments, however, antibodies (and their antigen binding fragments) will not cross-react with other antigens. Antibodies may also bind to other molecules in a way that is not immunospecific, such as to FcR receptors, by virtue of binding domains in other regions/domains of the molecule that do not involve the antigen recognition site, such as the Fc region.

As used herein, the terms “individual,” “host,” “subject,” “participant,” and “patient” are used interchangeably herein, and refer to a mammal, including, but not limited to, humans, rodents, such as mice and rats, and other laboratory animals. Thus, the methods and compositions described herein are applicable to both human and veterinary disease. In certain embodiments, subjects are “patients,” such as living humans that are receiving medical care for a disease or condition. This includes persons with no defined illness who are being investigated for signs of pathology.

As used herein, “inflammatory molecules” refer to molecules that result in inflammatory responses including, but not limited to, cytokines and metalloproteases such as including, but not limited to, IL-1β, TNF-α, TGF-beta, IFN-γ, IL-18, IL-17, IL-6, IL-23, IL-22, IL-21, and MMPs.

As used herein, “ligand” refers to a small molecule, peptide, polypeptide, or membrane associated or membrane-bound molecule, that is an agonist or antagonist of a receptor. “Ligand” also encompasses a binding agent that is not an agonist or antagonist and has no agonist or antagonist properties. By convention, where a ligand is membrane-bound on a first cell, the receptor usually occurs on a second cell. The second cell may have the same identity (the same name), or it may have a different identity (a different name), as the first cell. A ligand or receptor may be entirely intracellular, that is, it may reside in the cytosol, nucleus, or in some other intracellular compartment. The ligand or receptor may change its location, such as from an intracellular compartment to the outer face of the plasma membrane. The complex of a ligand and receptor is termed a “ligand receptor complex.” Where a ligand and receptor are involved in a signaling pathway, the ligand occurs at an upstream position and the receptor occurs at a downstream position of the signaling pathway.

As used herein, the term “isolated” means material that is substantially or essentially free from components that normally accompany it in its native state. In particular embodiments, the term “obtained” or “derived” is used synonymously with isolated.

As used herein, “management” or “controlling” one or more symptoms of effects of a disease or condition (e.g., cancer) refers to the use of the compositions or methods contemplated herein, to improve the quality of life for an animal by providing better control of tumor activity and clinical signs associates with cancer in a subject in need thereof.

As used herein the term “modulate” relates to a capacity to alter an effect, result, or activity (e.g., signal transduction). Such modulation can be agonistic or antagonistic. Antagonistic modulation can be partial (i.e., attenuating, but not abolishing) or it can completely abolish such activity (e.g., neutralizing). Modulation can include internalization of a receptor following binding of an antibody or a reduction in expression of a receptor on the target cell. Agonistic modulation can enhance or otherwise increase or enhance an activity (e.g., signal transduction). In a still further embodiment, such modulation can alter the nature of the interaction between a ligand and its cognate receptor so as to alter the nature of the elicited signal transduction. For example, the molecules can, by binding to the ligand or receptor, alter the ability of such molecules to bind to other ligands or receptors and thereby alter their overall activity. In some embodiments, such modulation will provide at least a 10% change in a measurable immune system activity, at least a 50% change in such activity, or at least a 2-fold, 5-fold, 10-fold, or at least a 100-fold change in such activity.

As used herein, the terms “percent sequence identity” and “% sequence identity” refer to the percentage of sequence similarity found by a comparison or alignment of two or more amino acid or nucleic acid sequences. Percent identity can be determined by a direct comparison of the sequence information between two molecules by aligning the sequences, counting the exact number of matches between the two aligned sequences, dividing by the length of the shorter sequence, and multiplying the result by 100. An algorithm for calculating percent identity is the Smith-Waterman homology search algorithm (see, e.g., Kann and Goldstein (2002) Proteins 48:367-376; Arslan, et al. (2001) Bioinformatics 17:327-337). As non-limiting examples, percent sequence identity can be about 10%, about 20%, about 30%, about 40%, about 50%, about 60%, about 70%, about 80%, about 90%, and any percentages in between.

As used herein, “peptide” refers to a short sequence of amino acids, where the amino acids are connected to each other by peptide bonds. A peptide may occur free or bound to another moiety, such as a macromolecule, lipid, oligo- or polysaccharide, and/or a polypeptide. Where a peptide is incorporated into a polypeptide chain, the term “peptide” may still be used to refer specifically to the short sequence of amino acids. A “peptide” may be connected to another moiety by way of a peptide bond or some other type of linkage. A peptide is at least two amino acids in length, wherein the maximal length is a function of custom or context.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

$$100 times the fraction W / Z ,$$

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, a “pharmaceutically acceptable excipient,” “pharmaceutically acceptable carrier,” or “diagnostically acceptable excipient” includes but is not limited to, sterile distilled water, saline, phosphate buffered solutions, amino acid-based buffers, or bicarbonate buffered solutions. An excipient selected and the amount of excipient used will depend upon the mode of administration. Administration comprises an injection, infusion, or a combination thereof. Additional pharmaceutically acceptable carriers include but are not limited to suitable carriers or diluents commonly used in the formulation art including aqueous or organic solvents or mixtures of solvents. These organic solvents may be found, for example, in Remington Pharmaceutical Sciences, 21^{st} Edition (2005). Other solvents and/or additives that may be used in the topical compositions include, but are not limited to, PEG ethers and PEG esters including, but not limited to, PEG esters of carboxylic acids and dicarboxylic acids and PEG esters of fatty acids, glycerol esters including triacetin, caprylic/capric triglycerides (Miglyol 812®) and the like; glycerol ethers including glycerol formal; propylene glycol dicaprylate/dicaprate (Miglyol 840®), lauryl lactate, triacetin, diisopropyl adipate (DIPA, also known as CERAPHYL 230), diisobutyl adipate, dimethyl isosorbide (DMI), acetyltributyl citrate, oleic acid; carboxylic acid esters including esters of diacids, ketones including acetone, methylisobutyl ketone (MIK), methyl ethyl ketone, and the like; acetonitrile, C₁-C₁₂ alcohols including benzyl alcohol, methanol, ethyl alcohol, isopropanol, and butanol; aromatic ethers such as anisole; amides including dimethylacetamide, monomethylacetamide and dimethylformamide; dimethyl sulfoxide (DMSO), ethylene glycol, propylene glycol, a glycol carbonate including, but not limited to, propylene carbonate and, butylene carbonate; 2-pyrrolidone, N-methylpyrrolidone, C₁-C₁₂ alkyl esters of carboxylic acids including butyl or octyl acetate and benzyl acetate; C₁-C₁₂ alkyl esters of dicarboxylic acids; aryl esters including benzyl benzoate, ethyl benzoate and the like; and diethyl phthalate, or a mixture of at least two of these solvents.

As used herein, the term “polypeptide” refers to a chain of amino acids of any length, regardless of modification (e.g., phosphorylation or glycosylation). The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, “prevent,” and similar words such as “prevented,” “preventing” etc., indicate an approach for preventing, inhibiting, or reducing the likelihood of the occurrence or recurrence of one or more symptoms or other effects of a disease or condition disclosed herein, such as a disease or condition like colorectal cancer, head and neck cancer, gastrointestinal cancer, gastroesophageal cancer, and other known diseases and conditions. For example, in embodiments that relate to treating cancer in a subject, “prevent,” and similar words such as “prevented,” “preventing” and the like indicate an approach for preventing, inhibiting, or reducing the likelihood of the occurrence of clinical signs and symptoms associated with cancer. It also refers to delaying the onset or recurrence of a disease or condition or delaying the occurrence or recurrence of the symptoms.

As used herein, the term “prophylactic agent” refers to an agent that can be used in the prevention of a disorder or disease prior to the detection of any symptoms of such disorder or disease. A “prophylactically effective” amount is the amount of prophylactic agent (e.g., protein, polypeptide, fragment thereof, etc.) sufficient to mediate such protection. A prophylactically effective amount may also refer to the amount of the prophylactic agent that provides a prophylactic benefit in the prevention of disease. Typically, but not necessarily, since a prophylactic dose is used in subjects prior to or at an earlier stage of disease, the prophylactically effective amount may be less than the therapeutically effective amount.

As used herein, “protein” generally refers to the sequence of amino acids comprising a polypeptide chain. Protein may also refer to a three-dimensional structure of the polypeptide. “Denatured protein” refers to a partially denatured polypeptide, having some residual three-dimensional structure or, alternatively, to an essentially random three-dimensional structure, as is the case in a totally denatured protein. Polypeptide variants can be produced by glycosylation, phosphorylation, sulfation, disulfide bond formation, deamidation, isomerization, cleaving points in signal or leader sequence processing, covalent and non-covalently bound cofactors, oxidized variants, and the like.

As used herein, “recombinant” when used with reference to a nucleic acid, cell, animal, virus, plasmid, vector, or the like, indicates modification by the introduction of an exogenous, non-native nucleic acid, alteration of a native nucleic acid, or by derivation in whole or in part from a recombinant nucleic acid, cell, virus, plasmid, or vector. Recombinant protein refers to a protein derived from a recombinant nucleic acid, virus, plasmid, vector, or the like.

As used herein, “sample” refers to a sample from a human, animal, placebo, or research sample, such as a cell, tissue, organ, fluid, gas, aerosol, slurry, colloid, or coagulated material. The “sample” may be tested in vivo, (i.e., without removal from the human or animal), or it may be tested in vitro. The sample may be tested after processing, such as by histological methods. “Sample” also refers to a cell comprising a fluid or tissue sample, or a cell separated from a fluid or tissue sample. “Sample” may also refer to a cell, tissue, organ, or fluid that is freshly taken from a human or animal, or to a cell, tissue, organ, or fluid that is processed or stored.

“Specifically” or “selectively” binds, when referring to a ligand/receptor, nucleic acid/complementary nucleic acid, antibody/antigen, or other binding pair (e.g., a cytokine to a cytokine receptor) indicates a binding reaction which is determinative of the presence of the protein in a heterogeneous population of proteins and other biologics. Thus, under designated conditions, a specified ligand binds to a particular receptor and does not bind in a significant amount to other proteins present in the sample. Specific binding can also mean, e.g., that the binding compound, nucleic acid ligand, antibody, or binding composition derived from the antigen-binding site of an antibody, of the contemplated method binds to its target with an affinity that is often at least 25% greater, more often at least 50% greater, most often at least 100% (2-fold) greater, normally at least ten times greater, more normally at least 20-times greater, and most normally at least 100-times greater than the affinity with any other binding compound.

The term “substantially,” as used in the context of binding or exhibited effect, is intended to denote that the observed effect is physiologically or therapeutically relevant. Thus, for example, a molecule is able to substantially block an activity of a ligand or receptor if the extent of blockage is physiologically or therapeutically relevant (for example if such extent is greater than 60% complete, greater than 70% complete, greater than 75% complete, greater than 80% complete, greater than 85% complete, greater than 90% complete, greater than 95% complete, or greater than 97% complete). Similarly, a molecule is said to have substantially the same immunospecificity and/or characteristic as another molecule, if such immunospecificities and characteristics are greater than 60% identical, greater than 70% identical, greater than 75% identical, greater than 80% identical, greater than 85% identical, greater than 90% identical, greater than 95% identical, or greater than 97% identical).

As used herein, the term “therapeutically effective amount” is defined as an amount of a reagent or pharmaceutical composition that is sufficient to induce a desired immune response specific for encoded heterologous antigens to show a patient benefit (e.g., to cause a decrease, prevention, or amelioration of the symptoms of the condition being treated). When the agent or pharmaceutical composition comprises a diagnostic agent, a “diagnostically effective amount” is defined as an amount that is sufficient to produce a signal, image, or other diagnostic parameter. Effective amounts of the pharmaceutical formulation will vary according to factors such as the degree of susceptibility of the individual, the age, gender, and weight of the individual, and idiosyncratic responses of the individual (U.S. Pat. No. 5,888,530).

As used herein, “treat,” “treating,” “treatment” and “therapeutic use” (with respect to a condition or a disease) is an approach for obtaining beneficial or desired results including and preferably clinical results. For purposes of this disclosure, beneficial or desired results with respect to a disease include, but are not limited to, one or more of improving a condition associated with a disease, curing a disease, lessening severity of a disease, delaying progression of a disease, alleviating one or more symptoms associated with a disease, increasing the quality of life of one suffering from a disease, and/or prolonging survival. Likewise, for purposes of this disclosure, beneficial or desired results with respect to a condition include, but are not limited to, one or more of improving a condition, curing a condition, lessening severity of a condition, delaying progression of a condition, alleviating one or more symptoms associated with a condition, increasing the quality of life of one suffering from a condition, and/or prolonging survival.

As used herein, “tumor microenvironment” or “TME” refers to the normal cells, molecules, fibroblasts, immune cells, and blood vessels that surround and feed a tumor cell. The tumor microenvironment also includes proteins produced by all of the cells present in the tumor that support the growth of the cancer cells.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gin: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

### II. Compositions

The present disclosure relates to combinations of one or more pharmaceutical compositions. The one or more pharmaceutical compositions may be combined into one administration or administered separately according to a variety of dosing and timing regimens, as described herein. A combination can be administered to a subject in need thereof, wherein the subject is experiencing advanced unresectable and/or metastatic Immune Checkpoint Inhibitor (ICI) refractory solid tumors or ICI naïve microsatellite stable/microsatellite instable (MSS/MSI) low solid tumors. A combination can be administered to a subject in need thereof, wherein one composition of the combination is an ICI (e.g. anti-PD-1 treatment) while a second composition of the combination shows an affinity for binding components of an extracellular matrix (ECM) in a tumor microenvironment (TME). The second composition may bind components of the ECM as to allow the ICI of the first composition to more efficiently target tumors. The combination then more efficiently treats, reduces, or kills tumors associated with cancer. The composition showing an infinity for binding components of the ECM may bind collagen. A combination can include but is not limited to a first pharmaceutical composition comprising Pembrolizumab and a second pharmaceutical composition comprising LAIR-2 or a LAIR-2 Fc fusion protein. The second pharmaceutical composition may particularly comprise NC410, a LAIR-2 Fc fusion protein. A combination may include additional compositions including but not limited to one or more compositions of LAIR-2 IgG1 fusion protein, LAIR-1, LAIR-1 Fc fusion proteins, humanized monoclonal antibodies (e.g. humanized monoclonal antibody against PD-1 (IgG4)), collagen-derived products (e.g. C4G, Pro-C3, Pro-C6, and the like), any combinations thereof, and other such components known in the art.

### A. Immune Checkpoint Pathways

The present disclosure is related to a combination of one or more compositions configured to effectively inhibit Programmed Cell Death Protein 1 (PD-1) pathways. Inhibition of the PD-1 pathway in the tumor microenvironment (TME) with an immune checkpoint inhibitor (e.g. Pembrolizumab) in conjunction with a protein configured to interact with components of the extracellular matrix (e.g. LAIR proteins) should promote immune cell activation coupled with extracellular matrix (ECM) remodeling, and further T cell infiltration into the TME, providing a novel and improved treatment approach for participants with ICI refractory advanced metastatic solid tumors regardless of MSI status or MSS or MSI-low advanced unresectable and/or metastatic solid tumors. The PD-1 receptor-ligand interaction is a major pathway hijacked by tumors to suppress immune control. The normal function of PD-1, expressed on the cell surface of activated T-cells under healthy conditions, is to down-modulate unwanted or excessive immune responses, including autoimmune reactions. PD-1 (encoded by the gene PDCD1) is an immunoglobulin (Ig) superfamily member related to cluster of differentiation 28 (CD28) and cytotoxic T-lymphocyte-associated protein 4 (CTLA-4) that has been shown to negatively regulate antigen receptor signaling upon engagement of its ligands (PD-L1 and/or PD-L2) (Okazaki, T., et al., “PD-1 immunoreceptor inhibits B cell receptor-mediated signaling by recruiting src homology 2-domain-containing tyrosine phosphatase 2 to phosphotyrosine”, Proc Natl Acad Sci U.S.A., 98:13866-71 (2001); Greenwald, R. J., et al., “The B7 family revisited”, Annu Rev Immunol., 23:515-48 (2005)).

Sequences for human PDCD1 are known in the art. For example, a consensus sequence for PDCD1 is:

MQIPQAPWPVVWAVLQLGWRPGWFLDSPDRPWNPPTFSPALLVVTEGDNAT
FTCSFSNTSESFVLNWYRMSPSNQTDKLAAFPEDRSQPGQDCRFRVTQLPN
GRDFHMSVVRARRNDSGTYLCGAISLAPKAQIKESLRAELRVTERRAEVPT
AHPSPSPRPAGQFQTLVVGVVGGLLGSLVLLVWVLAVICSRAARGTIGARR
TGQPLKEDPSAVPVFSVDYGELDFQWREKTPEPPVPCVPEQTEYATIVFPS
GMGTSSPARRGSADGPRSAQPLRPEDGHCSWPL
(SEQ ID NO: 1, UniProt accession number Q15116,
which is incorporated by reference in its
entirety).

The structure of murine PD-1 has been resolved (Zhang, 2004). PD-1 and its family members are type I transmembrane glycoproteins containing an Ig-variable-type (IgV-type) domain responsible for ligand binding and a cytoplasmic tail responsible for the binding of signaling molecules. The cytoplasmic tail of PD-1 contains two tyrosine-based signaling motifs—an immunoreceptor tyrosine-based inhibition motif, and an immunoreceptor tyrosine-based switch motif. Following T-cell stimulation, PD-1 recruits the tyrosine phosphatases, SHP-1 and SHP-2, to the immunoreceptor tyrosine-based switch motif within its cytoplasmic tail, leading to the dephosphorylation of effector molecules such as CD3 zeta (CD3ζ), protein kinase C-theta (PKCθ), and zeta-chain-associated protein kinase (ZAP70), which are involved in the CD3 T-cell signaling cascade (Okazaki, T., et al., “PD-1 immunoreceptor inhibits B cell receptor-mediated signaling by recruiting src homology 2-domain-containing tyrosine phosphatase 2 to phosphotyrosine”, Proc Natl Acad Sci U.S.A., 98:13866-71 (2001); Chemnitz, J. M., et al., “SHP-1 and SHP-2 associate with immunoreceptor tyrosine-based switch motif of programmed death 1 upon primary human T cell stimulation, but only receptor ligation prevents T cell activation”, J Immunol., 173:945-54 (2004); Sheppard, K. A., et al., “PD-1 inhibits T-cell receptor induced phosphorylation of the ZAP70/CD3zeta signalosome and downstream signaling to PKCtheta.”, FEBS Lett., 574:37-41 (2004); Riley, J. L., “PD-1 signaling in primary T cells”, Immunol Rev., 229:114-25 (2009)). The mechanism by which PD-1 down-modulates T-cell responses is similar to, but distinct from, that of CTLA-4, because both molecules regulate an overlapping set of signaling proteins (Parry, R. V., et al., “CTLA-4 and PD-1 receptors inhibit T-cell activation by distinct mechanisms”, Mol Cell Biol., 25:9543-53 (2005); Francisco, L. M., et al., “The PD-1 pathway in tolerance and autoimmunity”, Immunol Rev., 236:219-42 (2010)). As a consequence, the PD-1/PD-L1 pathway is an attractive target in the tumor microenvironment (TME) and an effective approach for cancer therapy.

### B. LAIR Proteins

The present disclosure is related to compositions comprising LAIR proteins. LAIR proteins can include an amino acid sequence of full-length LAIR-1 or LAIR-2 proteins, or a fragment or variant thereof, or a fusion protein thereof, including but not limited to LAIR-1 Fc fusion or LAIR-2 Fc fusion proteins. LAIR-2 Fc fusion proteins may include NC410. Compositions of LAIR proteins can be administered to a subject in need thereof in combination with one or more compositions of Pembrolizumab. Compositions of LAIR proteins can be administered simultaneously with the one or more compositions or separately. Compositions of LAIR proteins can be administered according to a variety of dosing and timing regimens, as described herein.

LAIR-1 inhibitory signaling may prevent autoimmune diseases such as lupus erythematosus, rheumatoid arthritis, autoimmune thyroid disease and atherosclerosis as well as contact hypersensitivity (Sun et al., 2014). Meanwhile, overexpression of LAIR-2 may promote autoimmunity through decoy binding of LAIR-1 ligands. LAIR-2 binding of LAIR-1 ligands can essentially reduce the cell surface cross-linking of LAIR-1, delimiting inhibitory signaling pathways leading to over-reactive immune function. Conversely, it hypothesized that increased levels of LAIR-2 may promote anti-tumor immunity through the same mechanism.

The presence of a collagen-dense extracellular matrix (ECM) is increasingly being recognized as a critical determinant of tumor responses to ICI therapy. Collagens can be secreted in the TME by cancer-associated fibroblasts (CAFs), cancer cells, and macrophages. LAIR-1 expressing cells localized to tumor microenvironments may be particularly suppressed through collagen cross-linking of LAIR-1 and subsequent inhibitory signaling. Interestingly, both collagen and C1q have been shown to limit or alter antigen-presenting cell (monocyte/macrophage/DC) differentiation and activation through LAIR-1. Studies have indicated that cross-linking LAIR-1 on NK cells that T cells can inhibit proliferation and function. Functioning as a physical barrier to immune cell infiltration into the tumor, a collagen-dense ECM has been shown to suppress antitumor immunity and to associate with PD-1/PD-L1 resistant tumors (Peng, D. H., et al., “Collagen promotes anti-PD-1/PD-L1 resistance in cancer through LAIR1-dependent CD8 (+) T cell exhaustion”, Nat Commun., 11:4520 (2020)).

Abnormalities in the ECM of the TME support tumor progression, lead to immune dysfunction, and provide targets for cancer therapeutics. Leukocyte-Associated Immunoglobulin-like Receptor (LAIR)-1 and LAIR-2 are members of the Leukocyte Receptor Complex (LRC) on human chromosome 19 (Lebbink, R. J., et al., “The soluble leukocyte-associated Ig-like receptor (LAIR)-2 antagonizes the collagen/LAIR-1 inhibitory immune interaction”, J Immunol., 180:1662-9 (2008); Lebbink, R. J., et al., “Identification of multiple potent binding sites for human leukocyte associated Ig-like receptor LAIR on collagens II and III”, Matrix Biol., 28:202-10 (2009); Olde Nordkamp, M. J., et al., “Enhanced secretion of leukocyte-associated immunoglobulin-like receptor 2 (LAIR-2) and soluble LAIR-1 in rheumatoid arthritis: LAIR-2 is a more efficient antagonist of the LAIR-1-collagen inhibitory interaction than is soluble; Olde Nordkamp, M. J., et al., “Leukocyte-associated Ig-like receptor-1 is a novel inhibitory receptor for surfactant protein D”, J Leukoc Biol, 96:105-11 (2014)). LAIR-1 is a well-described co-inhibitory receptor expressed on several subsets of immune cells, and functions to delimit immune responses (Afshar-Kharghan, V., “The role of the complement system in cancer”, J Clin Invest., 127:780-9 (2017); Pearce, O. M. T., et al., “Deconstruction of a Metastatic Tumor Microenvironment Reveals a Common Matrix Response in Human Cancers”, Cancer Discov., 8:304-19 (2018)). it has been observed that LAIR-1 expression is associated with suppressive immune cell populations in some cancers. A suppressive, but not stimulatory dendritic cell (DC) subpopulation, as well as suppressive macrophages express LAIR-1 in both mouse and human ovarian cancers, as shown in FIG. 1, indicating that blockade of LAIR-1 in ovarian cancer should reverse immune suppression (Flies, D. B., et al., “Immune checkpoint blockade reveals the stimulatory capacity of tumor-associated CD103(+) dendritic cells in late-stage ovarian cancer”, Oncoimmunology., 5: e1185583 (2016)).

### i. LAIR-1

Sequences for human LAIR-1 are known in the art. For example, a consensus sequence for LAIR-1a (isoform 1) is

MSPHPTALLGLVLCLAQTIHTQEEDLPRPSISAEPGTVIPLGSHVTFVCR
GPVGVQTFRLERESRSTYNDTEDVSQASPSESEARFRIDSVSEGNAGPYR
CIYYKPPKWSEQSDYLELLVKETSGGPDSPDTEPGSSAGPTQRPSDNSHN
EHAPASQGLKAEHLYILIGVSVVFLFCLLLLVLFCLHRQNQIKQGPPRSK
DEEQKPQQRPDLAVDVLERTADKATVNGLPEKDRETDTSALAAGSSQEVT
YAQLDHWALTQRTARAVSPQSTKPMAESITYAAVARH
(SEQ ID NO: 2, UniProtKB-Q6GTX8 (LAIR1_HUMAN)).

Amino acids 1-21 are a signal sequence, amino acids 22-165 (underlined) are an extracellular domain, amino acids 166-186 are a transmembrane domain, and amino acids 187-287 are a cytoplasmic domain. Amino acids 29-117 form an Ig-like C2-domain. Amino acids 249-254 and 279-284 form ITIM motif 1 and 2, respectively. LAIR-1b (also known as isoform 2) is missing amino acids 122-138 relative to SEQ ID NO:2. LAIR-1c (also known as isoform 3) is missing amino acids 23-23 and 122-138 relative to SEQ ID NO:2. LAIR-1d (also known as isoform 4) is missing amino acids 210-287 relative to SEQ ID NO:2.

As introduced above, an extracellular domain for human LAIR-1 can be

(SEQ ID NO: 3)
QEEDLPRPSISAEPGTVIPLGSHVTFVCRGPVGVQTERLERESRSTYNDT
EDVSQASPSESEARFRIDSVSEGNAGPYRCIYYKPPKWSEQSDYLELLVK
ETSGGPDSPDTEPGSSAGPTQRPSDNSHNEHAPASQGLKAEHLY,

or a fragment thereof. For example, the Ig-like C2-domain (underlined amino acids 8-96 of SEQ ID NO:3), or the region framed by the cysteines that form the disulfide bond between amino acids 49-101 of SEQ ID NO:2 (amino acids 28-80 of SEQ ID NO: 3, illustrated in italics).

Known variants and mutants of LAIR-1 include E63D, Y251F, and Y251F, relative to SEQ ID NO:2. Evidence shows that Y215F reduced tyrosine phosphorylation and loss of binding to PTPN6 and CSK as well as complete loss of inhibitory activity, as well as loss of phosphorylation and of inhibition of calcium mobilization when associated with F-281 (Xu, et al., J. Biol. Chem. 275:17440-17446 (2000), Verbrugge, et al., Int. Immunol., 15:1349-1358 (2003), Verbrugge, et al., Eur. J. Immunol., 36:190-198 (2006)). Y281F shows reduced tyrosine phosphorylation and loss of binding to PTPN6, and partial inhibition of cytotoxic activity.

Studies have demonstrated that components of the ECM serve as ligands for a collagen-binding inhibitory receptor LAIR-1. All collagens are composed of 3 polypeptide chains that are characterized by a repeating Gly-X-X′ sequence, where X is often proline and X′ frequently 4-R-hydroxyproline (Hyp, O) (Brondijk, Blood, 115(7): 1364-1373 (2010). The GPO triplets are an almost exclusive feature of collagens and allow the formation of the characteristic triplehelical collagen structure.

LAIR-1 ligands include several types of collagens, as well as ligands with collagen domains including complement component C1q, Mannan Binding Lectin (MBL) and Surfactant Protein D (SP-D) (Lebbink, R. J., et al., “The soluble leukocyte-associated Ig-like receptor (LAIR)-2 antagonizes the collagen/LAIR-1 inhibitory immune interaction”, J Immunol., 180:1662-9 (2008); Lebbink, R. J., et al., “Identification of multiple potent binding sites for human leukocyte associated Ig-like receptor LAIR on collagens II and III”, Matrix Biol., 28:202-10 (2009); Olde Nordkamp, M. J., et al., “Enhanced secretion of leukocyte-associated immunoglobulin-like receptor 2 (LAIR-2) and soluble LAIR-1 in rheumatoid arthritis: LAIR-2 is a more efficient antagonist of the LAIR-1-collagen inhibitory interaction than is solubl; Olde Nordkamp, M. J., et al., “Leukocyte-associated Ig-like receptor-1 is a novel inhibitory receptor for surfactant protein D”, J Leukoc Biol, 96:105-11 (2014)).

In cancer, it is hypothesized that LAIR-1 expression on several subsets of leukocytes prevents optimal immune responses by limiting both innate and adaptive immune functionality. LAIR-1 serves to suppress anti-tumor immunity through the inhibition of stimulatory signaling pathways. Specifically, LAIR-1 is a checkpoint and adhesion receptor on T cells that limits T cell activation and increases adhesion to collagens (Meyaard, L., “The inhibitory collagen receptor LAIR-1 (CD305)”, J Leukoc Biol., 83:799-803 (2008)).

In addition to its role in T cell function, LAIR-1 is also expressed on NK, monocyte, macrophage, dendritic cells and neutrophils and functions to delimit immune responses. Further, LAIR-1 expression has been shown to be associated with suppressive DC and macrophage subpopulations in both mouse and human ovarian cancers (Flies, D. B., et al., “Immune checkpoint blockade reveals the stimulatory capacity of tumor-associated CD103(+) dendritic cells in late-stage ovarian cancer”, Oncoimmunology., 5:e1185583 (2016)). Blockade of LAIR-1 in cancer should reduce inhibitory mechanisms to redirect myeloid cells toward promoting stimulatory responses, including T cell responses, against tumors. Overall, these data suggest that targeting the LAIR-1 pathway in cancer patients may be a rational approach to potentiate anti-tumor immunity.

It has been shown that certain tumors that have higher collagen deposition result in either inherent or acquired resistance to PD-1/PD-L1 blockade due to alternative immune suppression pathways and decreased total intra-tumoral CD8+ T cells (Peng, D. H., et al., “Collagen promotes anti-PD-1/PD-L1 resistance in cancer through LAIR1-dependent CD8(+) T cell exhaustion”, Nat Commun., 11:4520 (2020)). This phenomenon of collagen-induced CD8+ T cell immune suppression is due to over expression of LAIR-1 on immune cells, and enhanced collagen production in part driven by increased TGF-β signaling upon treatment with PD-1/PD-L1 blockade.

### ii. LAIR-2

LAIR-2 is a soluble homolog of LAIR-1 that binds to and out competes LAIR-1 binding to collagens and serves as a natural decoy to promote immune function. LAIR-2 is capable of blocking LAIR-1 functional interactions with ligands, resulting in improved immune function on multiple immune cell subsets. Given LAIR-2 binds with higher affinity to collagen than LAIR-1, overexpression of LAIR-2 results in blockade of LAIR-1 signaling, sensitizing resistant tumors to PD-1 blockade and markedly reducing tumor growth and metastasis.

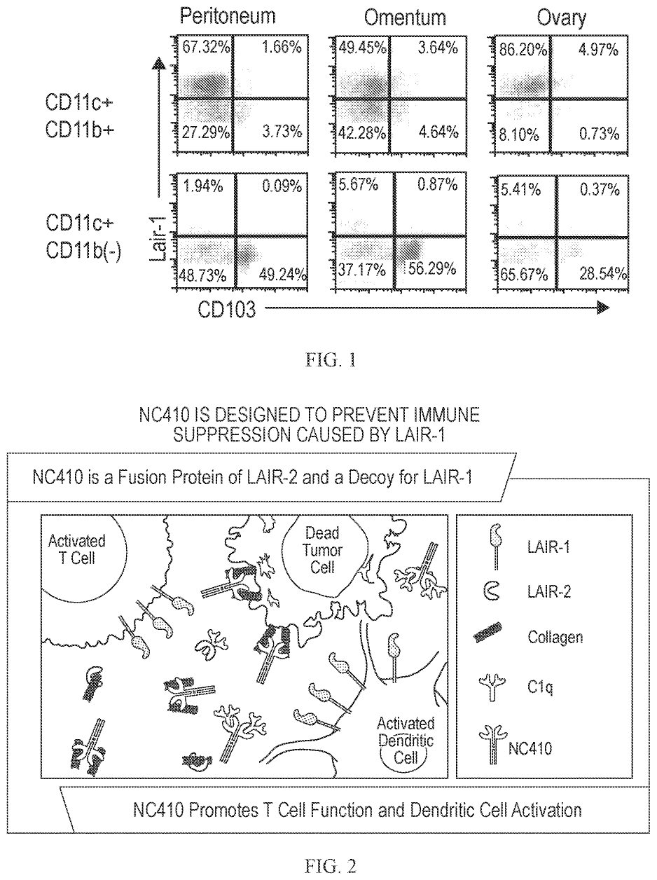

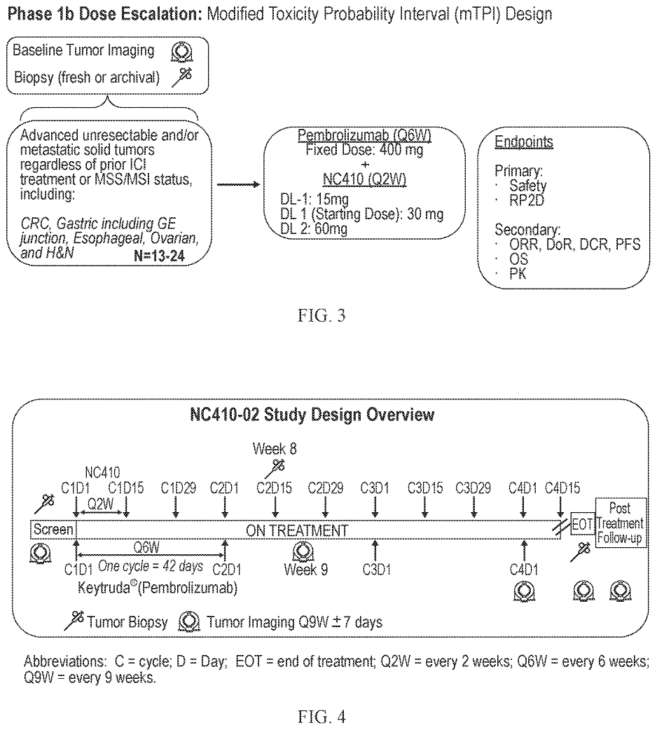

LAIR-2 is a secreted protein with 77.5% homology in the Ig-like C2 domain of extracellular region to the transmembrane protein LAIR-1 and serves as a natural, endogenous, secreted decoy for LAIR-1 produced primarily by activated T cells (Meyaard, L., “The inhibitory collagen receptor LAIR-1 (CD305)”, J Leukoc Biol., 83:799-803 (2008)). LAIR-2 is capable of blocking LAIR-1 functional interactions with ligands, resulting in improved immune function on multiple immune cell subsets, as shown in FIG. 2. Of note is the observation in which dysregulation of LAIR-1 ligands results in excessive production of collagens and complement C1q as well as altered forms of collagens that may exert strong inhibitory effects in the TME (Afshar-Kharghan, V., “The role of the complement system in cancer”, J Clin Invest., 127:780-9 (2017); Pearce, O. M. T., et al., “Deconstruction of a Metastatic Tumor Microenvironment Reveals a Common Matrix Response in Human Cancers”, Cancer Discov., 8:304-19 (2018)). Thus, disrupting the interaction of LAIR-1 ligands with LAIR-1 and interrupting the inhibitory effects of the TME utilizing a LAIR-2 based therapy is a novel approach to cancer therapy.

Sequences for human LAIR-2 are known in the art. For example, a consensus sequence for LAIR-2a (isoform 1) is

MSPHLTALLGLVLCLAQTIHTQEGALPRPSISAEPGTVISPGSHVTFMCR
GPVGVQTFRLEREDRAKYKDSYNVFRLGPSESEARFHIDSVSEGNAGLYR
CLYYKPPGWSEHSDFLELLVKESSGGPDSPDTEPGSSAGTVPGTEASGED
AP
(SEQ ID NO: 4, UniProtKB-Q6ISS4 (LAIR2_HUMAN)).

Amino acids 1-21 are a signal sequence, amino acids 22-152 (underlined) are the Leukocyte-associated immunoglobulin-like receptor 2 domain. Amino acids 29-117 form an Ig-like C2-domain. LAIR-2b (also known as isoform 2) is missing amino acids 122-138 relative to SEQ ID NO:4. As introduced above, a Leukocyte-associated immunoglobulin-like receptor 2 domain for human LAIR-2 can be

(SEQ ID NO: 5)
QEGALPRPSISAEPGTVISPGSHVTFMCRGPVGVQTFRLEREDRAKYKD
SYNVFRLGPSESEARFHIDSVSEGNAGLYRCLYYKPPGWSEHSDFLELL
VKESSGGPDSPDTEPGSSAGTVPGTEASGFDAP,

or a fragment thereof. For example, the Ig-like C2-domain (underlined amino acids 8-96 of SEQ ID NO:5), or the region framed by the cysteines that form the disulfide bond between amino acids 49-101 of SEQ ID NO:1 (amino acids 28-80 of SEQ ID NO: 5, illustrated in italics).

Known variants and mutants of LAIR-2 include G78S, H87R, and F115Y, relative to SEQ ID NO:4.

LAIR-2 in humans has been shown to bind collagen and SP-D with higher affinity than LAIR-1 (Meyaard, L., “The inhibitory collagen receptor LAIR-1 (CD305)”, J Leukoc Biol., 83:799-803 (2008), J. Leukoc. Biol. 83:799-803). Dr. Linde Meyaard has demonstrated that LAIR-2 also binds C1q and mannose-binding lectin (MBL), both of which contain collagen-like domains (Olde Nordkamp et al., J. Innate Immun., 2014, 6 (3): 284-92). This finding confirms evidence by Son et al that LAIR-2 binds C1q (Son et al., 2012, Proc. Natl. Acad. Sci. USA 109:E3160-3167). While collagens and C1q are ubiquitously expressed, SP-D is largely restricted to mucosal surfaces (lung alveolar surface and GI tract) where it functions as a first-line innate defense against pathogens (Herias et al., 2007, Mol. Immunol. 44:3324-3332).

An exemplary alignment of the human LAIR-1 and human LAIR-2 extracellular domains is shown below:

| Score | Expect | Method | Identities | Positives | Gaps |
|---|---|---|---|---|---|
| 194 bits(493) | 4e−70 | Compositional matrix adjust. | 95/118(81%) | 103/118(87%) | 0/118(0%) |

| | |
|---|---|
| Query 1 | QEEDLPRPSISAEPGTVIPLGSHVTFVCRGPVGVQTERLERESRSTYNDTEDVSQASPSE 60 |
| | QE LPRPSISAEPGTVI GSHVTF+CRGPVGVQTFRLERE R+ Y D+ +V * PSE |
| Sbjct 1 | QEGALPRPSISAEPGTVISPGSHVTFMCRGPVGVQTFRLEREDRAKYKDSYNVFRLGPSE 66 |
| Query 61 | SEARFRIDSVSEGNAGPYRCIYYKPPKWSEQSDYLELLVKETSGGPDSPDTEPGSSAG 118 |
| | SEARF IDSVSEGNAG YRC+YYKPP WSE SD+LELLVKE+SGGPDSPDTEPGSSAG |
| Sbjct 61 | SEARFHIDSVSEGNAGLYRCLYYKPPGWSEHSDFLFLLVKESSGGPDSPDTEPGSSAG 118 |

Query is SEQ ID NO:2 and Sbjct is SEQ ID NO:4.

### III. NC410

NC410 is a dimeric form of the LAIR-2 protein fused to a human Fc domain of the immunoglobulin (Ig) subtype IgG1. NC410 targets tumor collagen to reverse LAIR-1-mediated immunosuppression, as well as induce ECM remodeling to promote immune cell infiltration and function in the TME, as shown in FIG. 2.

Pre-clinical studies in mouse models (HT-29, P815) with NC410 have been shown to enhance T cell expansion (both CD4+ and CD8+ cells), increase production of IFN-g and granzyme B, and anti-tumor effect in dose-dependent fashion. Because tumor-associated collagen induces CD8+ T cell exhaustion through LAIR-1-SHP-1 signaling, overexpression of LAIR-2 to inhibit LAIR-1 binding to collagen, resulted in reduction of tumor growth in a lung tumor model. Moreover, when LAIR-2 overexpression was combined with anti-PD-1 treatment growth and metastasis were significantly reduced within 1 week of treatment and was sustained throughout the course of treatment (Peng, D. H., et al., “Collagen promotes anti-PD-1/PD-L1 resistance in cancer through LAIR1-dependent CD8(+) T cell exhaustion”, Nat Commun., 11:4520 (2020)). Furthermore, NC410 with anti-PD-1 inhibitor has been shown consistently to reduce tumor burden.

NC410 has been studied in a variety of in vitro and in vivo systems to support its use as an investigational drug in oncology. These studies have demonstrated enhanced immune activity in mechanistic studies and tumor models. NC410 binds to LAIR-1 ligands including collagen, C1q, MBL and SP-D with high avidity and block the interaction of LAIR-1 to its ligands. NC410 reverses the inhibitory effects of collagen on the Lipopolysaccharide (LPS)-induced NF KB and Interferon signaling, important signaling pathways leading to immune cell activation. NC410 promotes primary monocyte activation and differentiation toward a stimulatory macrophage phenotype. NC410 enhances human T cell expansion and activation in a dose-dependent manner, which correlates with anti-tumor efficacy mouse P815 and human HT29 tumor models and chemokines such as CXCL10, CXCL11 and CXCL12 productions. NC410 promotes T cell dependent cytokine and chemokine production in the tumor microenvironment that correlates with tumor control and promotes tumor remodeling as evidenced by changes in levels of collagen degradative products.

### A. Proteins and Polypeptide Compositions

The ECM-binding agent and ICI can be a protein, polypeptide, or fusion protein. For example, the ECM-binding agent and ICI can be an isolated or recombinant protein or polypeptide, or functional fragment, variant, or fusion protein thereof of LAIR-2 or Pembrolizumab, as described above.

The protein or polypeptide, or functional fragment, variant, or fusion protein thereof can be an agonist or an antagonist. For example, in some embodiments an antagonist of LAIR-2 is a LAIR-1 or LAIR-2 polypeptide or a fragment or fusion protein thereof that binds to a ligand of LAIR-2. The polypeptide can be a soluble fragment, for example the extracellular domain of LAIR-2, or a functional fragment thereof, or a fusion protein thereof. In some embodiments, a soluble ligand of LAIR-2 may serve as an agonist, increasing signal transduction through LAIR-2.

The activity (i.e., agonist or antagonist) of a protein or polypeptide of LAIR-2, or any fragment, variant or fusion protein thereof can be determined using functional assays that are known in the art, and include the assays discussed below. Typically, the assays include determining if the protein, polypeptide or fragment, variant or fusion protein thereof increases (i.e., agonist) or decreases (i.e., antagonist) signaling through the LAIR-2 receptor. In some embodiments the assay includes determining if the protein, polypeptide or fragment, variant, or fusion protein thereof increases (i.e., agonist) or decreases (i.e., antagonist) the immune response (i.e., costimulatory or coinhibitory) associated with LAIR-2. Typically, the assays include determining if the protein, polypeptide or fragment, variant, or fusion protein thereof increases (i.e., agonist) or decreases (i.e., antagonist) signaling through LAIR-2. In some embodiments the assay includes determining if the protein, polypeptide or fragment, variant, or fusion protein thereof decreases (i.e., agonist) or increases (i.e., antagonist) an immune response negatively regulated by LAIR-2. In some embodiments the assay includes determining if the protein, polypeptide or fragment, variant, or fusion protein thereof increases (i.e., antagonist) the apoptosis and differentiation of acute myeloid leukemia cells and acute lymphoblastic leukemia cells resulting in reduced self-renewal capacity of AML and ALL stem cells.

Nucleic acid and polypeptide sequences for LAIR-1 and LAIR-2 are known in the art and exemplary protein and peptide sequences are provided above. The sequences can be used, as discussed in more detail below, by one of skill in the art to prepare any protein or polypeptide of LAIR-1 or LAIR-2, or any fragment, variant, or fusion protein thereof. Generally, the proteins, polypeptides, fragments, variants, and fusions thereof of LAIR-1 and LAIR-2 are expressed from nucleic acids that include sequences that encode a signal sequence. The signal sequence is generally cleaved from the immature polypeptide to produce the mature polypeptide lacking the signal sequence. The signal sequence can be replaced by the signal sequence of another polypeptide using standard molecule biology techniques to affect the expression levels, secretion, solubility, or other property of the polypeptide. LAIR-1 and LAIR-2 both with and without a signal sequence are disclosed. It is understood that in some cases, the mature protein as it is known or described in the art, i.e., the protein sequence without the signal sequence, is a putative mature protein. During normal cell expression, a signal sequence can be removed by a cellular peptidase to yield a mature protein. The sequence of the mature protein can be determined or confirmed using methods that are known in the art.

### i. Fragments

As used herein, a fragment of LAIR-1 or LAIR-2 refers to any subset of the polypeptide that is at least one amino acid shorter than full length protein. Useful fragments include those that retain the ability to bind to their natural ligand or ligands. A polypeptide that is a fragment of any full-length LAIR-1 or LAIR-2 typically has at least 20 percent, 30 percent, 40 percent, 50 percent, 60 percent, 70 percent, 80 percent, 90 percent, 95 percent, 98 percent, 99 percent, 100 percent, or even more than 100 percent of the ability to bind its natural ligand respectively as compared to the full-length protein.

Fragments of LAIR-1 and LAIR-2 include cell free fragments. Cell free polypeptide can be fragments of full-length, transmembrane, polypeptides that may be shed, secreted or otherwise extracted from the producing cells. Cell free fragments of polypeptides can include some or all of the extracellular domain of the polypeptide, and lack some or all of the intracellular and/or transmembrane domains of the full-length protein. In one embodiment, polypeptide fragments include the entire extracellular domain of the full-length protein. In other embodiments, the cell free fragments of the polypeptides include fragments of the extracellular domain that retain biological activity of full-length protein. The extracellular domain can include 1, 2, 3, 4, or 5 contiguous amino acids from the transmembrane domain, and/or 1, 2, 3, 4, or 5 contiguous amino acids from the signal sequence. Alternatively, the extracellular domain can have 1, 2, 3, 4, 5 or more amino acids removed from the C-terminus, N-terminus, or both. In some embodiments the extracellular domain is the only functional domain of the fragment (e.g., the ligand binding domain).

### ii. Variants

Variants of LAIR-1 and LAIR-2, and fragments thereof are also provided. In some embodiments, the variant is at least 50, 60, 70, 80, 85, 90, 95, 96, 97, 98, or 99 percent identical to any one of SEQ ID NO:2-5. Useful variants include those that increase biological activity, as indicated by any of the assays described herein, or that increase half-life or stability of the protein. The protein and polypeptides of LAIR-1 or LAIR-2, and fragments, variants, and fusion proteins thereof can be engineered to increase biological activity. For example, in some embodiments, a LAIR-2 polypeptide, protein, or fragment, variant or fusion thereof has been modified with at least one amino acid substitution, deletion, or insertion that increases a function thereof.

Other variants are those that are engineered to selectively bind to one or more type of LAIR-1 and/or LAIR-2 ligands versus other LAIR-1 and/or LAIR-2 ligands. For example, the variants can be engineered to bind preferentially to one or more collagens, SP-D, C1q or MBL, or a specific combination thereof. Preferential binding refers to binding that is at least 10%, 20%, 30%, 40%, 50%, 60%, 70%, 80%, 90%, 95%, or greater for one type of ligand over another type of ligand.

Still other variants can be engineered to have reduced binding to one ligand compared to another. These variants can be used in combination with variants having stronger binding properties to modulate the immune response with a moderate impact.

In still other embodiments, the variants can be engineered to have reduced binding to one or more collagen binds sites relative others. As discussed in Brondijk, et al., Blood, 18 (115): 1364-73 (2010), mutation of residues with LAIR-1 can have differential effect on binding to different collagen ligands. For example, adhesion to immobilized collagens I, III, and IV was significantly reduced in the R59A, E61A, R65A, and E111A mutants, although the magnitude of the effect depended on the type of collagen tested. In addition, adhesion was somewhat reduced for mutants R62A and N69A. In some embodiments, the variant is mutated at one or more of R59, E61, R62, E63, R65, S66, Y68, N69, 1102, R100, W109, E111, Q112, and Y115 relative to SEQ ID NO:2. In some embodiments, the variant is mutated at one or more of R59, E61, R65, E111, R62A, and N69A. In particular embodiments, the mutation(s) is substitution with an alanine.

Finally, variant polypeptides can be engineered to have an increased half-life relative to wildtype. These variants typically are modified to resist enzymatic degradation. Exemplary modifications include modified amino acid residues and modified peptide bonds that resist enzymatic degradation. Various modifications to achieve this are known in the art. The variants can be modified to adjust for effects of affinity for the receptor on the half-life of proteins, polypeptides, fragments, or fusions thereof at serum and endosomal pH.

### iii. Fusion Proteins

Fusion polypeptides have a first fusion partner comprising all or a part of a polypeptide LAIR-1 or LAIR-2 fused to a second polypeptide directly or via a linker peptide sequence that is fused to the second polypeptide. The fusion proteins optionally contain a domain that functions to dimerize or multimerize two or more fusion proteins. The peptide/polypeptide linker domain can either be a separate domain, or alternatively can be contained within one of the other domains (first polypeptide or second polypeptide) of the fusion protein. Similarly, the domain that functions to dimerize or multimerize the fusion proteins can either be a separate domain, or alternatively can be contained within one of the other domains (first polypeptide, second polypeptide or peptide/polypeptide linker domain) of the fusion protein. In one embodiment, the dimerization/multimerization domain and the peptide/polypeptide linker domain are the same.

Fusion proteins disclosed herein are of formula I:

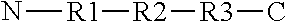

wherein “N” represents the N-terminus of the fusion protein, “C” represents the C-terminus of the fusion protein. In some embodiments, “R1” is a polypeptide or protein of LAIR-1 or Liar-2, or fragment or variant thereof, “R2” is an optional peptide/polypeptide linker domain, and “R3” is a second polypeptide. Alternatively, R3 may be a polypeptide or protein of LAIR-1 or LAIR-2, or fragment or variant thereof and R1 may be a second polypeptide. In some embodiments, the LAIR-1 or LAIR-2 polypeptide is the extracellular domain or a fragment thereof such as the Ig-like C2-domain, or the region framed by the cysteines that form a disulfide bond as discussed above.

Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric.

In some embodiments, the fusion protein includes the extracellular domain of LAIR-1 or LAIR-2, or a fragment or variant thereof, fused to an Ig Fe region. Recombinant Ig fusion proteins can be prepared by fusing the coding region of the extracellular domain of an extracellular domain or a fragment or variant thereof to the Fc region of human IgG1, IgG2, IgG3 or IgG4 or mouse IgG2a, or other suitable Ig domain, as described previously (Chapoval, et al., Methods Mol. Med., 45:247-255 (2000)).

### iv. Exemplary Fusion Proteins

Exemplary fusion proteins are provided below. The signal sequence is indicated by double underlining, the LAIR-2 extracellular domain by single underlining, and the Ig domain by italics. The signal sequence is typically removed in the mature protein. Additionally, signal peptides from other polypeptides or organisms can be used (e.g., substituted) to enhance the secretion of the fusion protein from a host during manufacture.

In some embodiments, human LAIR2-hIg fusion protein (hIgG1) (hLAIR2.hG1) has at least 80%, 85%, 90%, 95%, 99%, or 100% sequence identity to the amino acid sequence:

(SEQ ID NO: 6)
MEWSWVFLFELSVTTGVHSQEGALPRPSISAEPGTVISPGSHVTFMCRG
PVGVQTFRLEREDRAKYKDSYNVFRLGPSESEARFHIDSVSEGNAGLYRC
LYYKPPGWSEHSDFLELLVKESSGGPDSPDTEPGSSAGTVPGTEASGEDA
PDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQ
GNVFSCSVMHEALHNHYTQKSLSLSPG,

with or without the signal sequence.

SEQ ID NO:6 without the signal sequence is

(SEQ ID NO: 7)
QEGALPRPSISAEPGTVISPGSHVTFMCRGPVGVQTFRLEREDRAKYKDS
YNVFRLGPSESEARFHIDSVSEGNAGLYRCLYYKPPGWSEHSDELELLVK
ESSGGPDSPDTEPGSSAGTVPGTEASGFDAPDKTHTCPPCPAPELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQK
SLSLSPG.

Human LAIR2-hIg fusion protein (hIgG1) (hLAIR2.hG1) can be an antagonist for LAIR-1 signaling by serving as a decoy for LAIR-1 ligands, and can be utilized for the treatment of cancer or an infectious disease.

In some embodiments, human LAIR2.mIg fusion protein (mIgG2a) has at least 80%, 85%, 90%, 95%, 99%, or 100% sequence identity to the amino acid sequence:

(SEQ ID NO: 8)
MEWSWVELFELSVTTGVHSQEGALPRPSISAEPGTVISPGSHVTFMCRG
PVGVQTFRLEREDRAKYKDSYNVFRLGPSESEARFHIDSVSEGNAGLYRC
LYYKPPGWSEHSDFLELLVKESSGGPDSPDTEPGSSAGTVPGTEASGFDA
PEPRGPTIKPCPPCKCPAPNLLGGPSVFIFPPKIKDVLMISLSPIVTCVV
VDVSEDDPDVQISWFVNNVEVHTAQTQTHREDYNSTLRVVSALPIQHQDW
MSGKEFKCKVNNKDLPAPIERTISKPKGSVRAPQVYVLPPPEEEMTKKQV
TLTCMVTDFMPEDIYVEWTNNGKTELNYKNTEPVLDSDGSYFMYSKLRVE
KKNWVERNSYSCSVVHEGLHNHHTTKSFSRTPG,

with or without the signal sequence.

SEQ ID NO:8 without the signal sequence is

(SEQ ID NO: 9)
QEGALPRPSISAEPGTVISPGSHVTFMCRGPVGVQTFRLEREDRAKYKDS
YNVFRLGPSESEARFHIDSVSEGNAGLYRCLYYKPPGWSEHSDFLELLVK
ESSGGPDSPDTEPGSSAGTVPGTEASGEDAPEPRGPTIKPCPPCKCPAPN
LLGGPSVFIFPPKIKDVLMISLSPIVTCVVVDVSEDDPDVQISWFVNNVE
VHTAQTQTHREDYNSTLRVVSALPIQHQDWMSGKEFKCKVNNKDLPAPIE
RTISKPKGSVRAPQVYVLPPPEEEMTKKQVTLTCMVTDFMPEDIYVEWTN
NGKTELNYKNTEPVLDSDGSYFMYSKLRVEKKNWVERNSYSCSVVHEGLH
NHHTTKSFSRTPG.

Human LAIR2.mIg fusion protein (mIgG2a) can be used to generate antagonistic anti-LAIR2 (e.g., mAb, or fragments thereof), which can be used for the treatment of autoimmune diseases.

### IV. Immune Checkpoint Inhibitors

### A. PD-1 Antagonist

In some embodiments, LAIR-2-Fc is co-administered with a PD-1 receptor antagonist. Programmed Death-1 (PD-1) is a member of the CD28 family of receptors that delivers a negative immune response when induced on T cells. Contact between PD-1 and one of its ligands (B7-H1 or B7-DC) induces an inhibitory response that decreases T cell multiplication and/or the strength and/or duration of a T cell response. Suitable PD-1 antagonists are described in U.S. Pat. Nos. 8,114,845, 8,609,089, and 8,709,416, which are specifically incorporated by reference herein in their entities, and include compounds or agents that either bind to and block a ligand of PD-1 to interfere with or inhibit the binding of the ligand to the PD-1 receptor, or bind directly to and block the PD-1 receptor without inducing inhibitory signal transduction through the PD-1 receptor.

In some embodiments, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

It is believed that PD-1 signaling is driven by binding to a PD-1 ligand (such as B7-H1 or B7-DC) in close proximity to a peptide antigen presented by major histocompatibility complex (MHC) (see, for example, Freeman, Proc. Natl. Acad. Sci. U. S. A, 105:10275-10276 (2008)). Therefore, proteins, antibodies or small molecules that prevent co-ligation of PD-1 and TCR on the T cell membrane are also useful PD-1 antagonists.

In some embodiments, the PD-1 receptor antagonists are small molecule antagonists or antibodies that reduce or interfere with PD-1 receptor signal transduction by binding to ligands of PD-1 or to PD-1 itself, especially where co-ligation of PD-1 with TCR does not follow such binding, thereby not triggering inhibitory signal transduction through the PD-1 receptor.

Other PD-1 antagonists contemplated by the methods of this invention include antibodies that bind to PD-1 or ligands of PD-1, and other antibodies.

Suitable anti-PD-1 antibodies include, but are not limited to, those described in the following publications (the contents of which are each incorporated herein in its entirety):
PCT/IL03/00425 (Hardy et al., WO/2003/099196),
PCT/JP2006/309606 (Korman et al., WO/2006/121168),
PCT/US2008/008925 (Li et al., WO/2009/014708),
PCT/JP03/08420 (Honjo et al., WO/2004/004771),
PCT/JP04/00549 (Honjo et al., WO/2004/072286),
PCT/IB2003/006304 (Collins et al., WO/2004/056875),
PCT/US2007/088851 (Ahmed et al., WO/2008/083174),
PCT/US2006/026046 (Korman et al., WO/2007/005874),
PCT/US2008/084923 (Terrett et al., WO/2009/073533),
Berger et al., Clin. Cancer Res., 14:30443051 (2008).

A specific example of an anti-PD-1 antibody is an antibody described in Kosak, US20070166281 (pub. 19 Jul. 2007) at par. 42), a human anti-PD-1 antibody, which in some embodiments is administered at a dose of 3 mg/kg.

Exemplary anti-B7-H1 antibodies include, but are not limited to, those described in the following publications:
PCT/US06/022423 (WO/2006/133396, pub. 14 Dec. 2006)
PCT/US07/088851 (WO/2008/083174, pub. 10 Jul. 2008)
US 2006/0110383 (pub. 25 May 2006)

A specific example of an anti-B7-H1 antibody is an antibody described (WO/2007/005874, published 11 Jan. 2007)), a human anti-B7-H1 antibody.

Additional anti-PD-1 and anti-B7-H1 antibodies are disclosed in 2014/0044738, which is specifically incorporated by reference herein in its entirety.

For anti-B7-DC antibodies see U.S. Pat. Nos. 7,411,051, 7,052,694, 7,390,888, and U.S. Published Application No. 2006/0099203.

Other exemplary PD-1 receptor antagonists include, but are not limited to B7-DC polypeptides, including homologs and variants of these, as well as active fragments of any of the foregoing, and fusion proteins that incorporate any of these. In some embodiments, the fusion protein includes the soluble portion of B7-DC coupled to the Fc portion of an antibody, such as human IgG, and does not incorporate all or part of the transmembrane portion of human B7-DC.

The PD-1 antagonist can also be a fragment of a mammalian B7-H1, for example from mouse or primate, such as a human, wherein the fragment binds to and blocks PD-1 but does not result in inhibitory signal transduction through PD-1. The fragments can also be part of a fusion protein, for example an Ig fusion protein.

Other useful polypeptides PD-1 antagonists include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al., Immunity, Vol. 27, pp. 111-122, (2007)). Such fragments also include the soluble ECD portion of the PD-1 protein that includes mutations, such as the A99L mutation, that increases binding to the natural ligands (Molnar et al., PNAS, 105:10483-10488 (2008)). B7-1 or soluble fragments thereof, which can bind to the B7-H1 ligand and prevent binding to the endogenous PD-1 receptor, thereby preventing inhibitory signal transduction, are also useful.

PD-1 and B7-H1 anti-sense nucleic acids, both DNA and RNA, as well as siRNA molecules can also be PD-1 antagonists. Such anti-sense molecules prevent expression of PD-1 on T cells as well as production of T cell ligands, such as B7-H1, PD-L1 and/or PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al., J. Clin. Invest. 119 (8): 2231-2244 (2009), are readily taken up by cells that express PD-1 as well as ligands of PD-1 and reduce expression of these receptors and ligands to achieve a decrease in inhibitory signal transduction in T cells, thereby activating T cells.

### B. Pembrolizumab

Several monoclonal antibodies that inhibit the interaction between PD-1 and one or both of its ligands PD-L1 and PD-L2 have been approved for treating cancer. Pembrolizumab is a potent humanized immunoglobulin G4 (IgG4) monoclonal antibody (mAb) with high specificity of binding to the programmed cell death 1 (PD-1) receptor, thus inhibiting its interaction with programmed cell death ligand 1 (PD-L1) and programmed cell death ligand 2 (PD-L2). Based on preclinical in vitro data, Pembrolizumab has high affinity and potent receptor blocking activity for PD-1. Pembrolizumab has an acceptable preclinical safety profile and is in clinical development as an intravenous (IV) immunotherapy for advanced malignancies. Keytruda® (Pembrolizumab) is indicated for the treatment of patients across a number of indications.

The present disclosure is related to compositions comprising immune checkpoint inhibitors (ICI). As a non-limiting example, Pembrolizumab is an ICI known to inhibit PD-1 pathways. Compositions of Pembrolizumab can be administered to a subject in need thereof in combination with one or more compositions capable of binding components of the extracellular matrix (ECM) of a tumor microenvironment (TME), such as collagen. As a non-limiting example, LAIR-2 Fc fusion proteins (e.g., NC410) may bind collagen. Compositions of Pembrolizumab can be administered simultaneously with the one or more compositions or separately. Compositions of Pembrolizumab can be administered according to a variety of dosing and timing regimens, as described herein.

Several other checkpoint inhibitors have gained substantial attention in cancer treatment during the last decade due to the durability of the responses and increased survival benefit. Some favorable factors emerge as predictors of response to ICI including but not limited to the presence and activation status of tumor infiltrating T cells, expression of PD-L1 or high tumor mutational burden (TMB). Presence of multiple neoantigens originating from highly mutated tumors leads to increase in tumor infiltrating T cells favoring ICI responses. Similarly, Mis-matched Repair Deficient (dMMR) tumors have 10 to 100 folds increase in somatic mutations compared to pMMR (Mis-matched Repair Proficient) tumors. dMMR colorectal cancer (CRC) and dMMR non-CRC tumors have excellent response to Pembrolizumab when there was no response to Pembrolizumab in pMMR CRC (Le, D. T., et al., “PD-1 Blockade in Tumors with Mismatch-Repair Deficiency”, N Engl J Med., 372:2509-20 (2015)). Subsequently, the FDA has approved Pembrolizumab in unresectable or metastatic, microsatellite instability-high (MSI-H) or mismatch repair deficient (dMMR) solid tumors that have progressed following prior treatment and who have no satisfactory alternative treatment options. This favorable response to ICI is hypothesized to be due to abundance of neoantigens with accompanying immune cell infiltration in the TME. In contrast, response to ICI in patients with MSS/MSI-L/pMMR tumors with low mutational load and absence of “immune-competent” TME, is disappointing. Therefore, there is high unmet medical need to explore new strategies to enhance responses to ICI in MSS/MSI-L tumors.

Furthermore, only 30% of patients with advanced cancers of certain types could benefit from ICI monotherapy or in combination with chemotherapy or other agents and the majority lost responses due to a variety of resistant mechanisms including but not limited to emergence of T-regs, myeloid derived suppressor cells (MDSC), M2 macrophages, TGF-β driven collagens that promote anti-PD-1/PD-L1 resistance and through LAIR-1-mediated immunosuppression including preventing CD8+ and CD4+ T cell proliferation and activation.

### V. Therapeutic Compositions

### A. Administration Regimens

The compositions below are to be understood as exemplary compositions related to the present disclosure. Such are not intended to be limiting of the scope of the present disclosure.

The compositions described herein can be administered to a subject in need thereof, either alone or in combination with a pharmaceutically acceptable excipient and/or carrier, in an amount sufficient to induce an appropriate anti-tumor response. Administration can include injection, infusion, other methods disclosed herein, and other methods known in the art. Administration includes but is not limited to intravenous, intramuscular, subcutaneous, and the like. The response can comprise, without limitation, specific immune response, non-specific immune response, both specific and non-specific response, innate response, primary immune response, adaptive immunity, secondary immune response, memory immune response, immune cell activation, immune cell proliferation, immune cell differentiation, and cytokine expression.

The invention provides a method of providing an anti-tumor immunity in a mammal by administering to a mammal an effective amount of a combination therapy. The combination therapy, as described herein, comprises a first composition of an immune checkpoint inhibitor (ICI) and a second composition of the combination therapy shows an affinity for binding components of an extracellular matrix (ECM) in a tumor microenvironment (TME). Effective amounts of a combination therapy can be determined by one of skill in the art with consideration of individual differences in age, weight, tumor size, extent of infection or metastasis, and condition of the patient (i.e. subject). It can generally be stated that compositions of a combination therapy can be administered simultaneously or separately subject to the same or different dosing and timing regimens, as described herein. Combination therapies may also be administered multiple times at these dosages. The combination therapy can be administered by using infusion techniques that are commonly known in immunotherapy (Rosenberg, et al., New Eng. J. of Med., 1988). The optimal dosage and treatment regime for a particular patient can readily be determined by one skilled in the art of medicine by monitoring the patient for signs of disease and adjusting the treatment accordingly. An effective amount for a particular patient may vary depending on factors such as the condition being treated, the overall health of the patient, the route and dose of administration and the severity of side effects. Guidance for methods of treatment and diagnosis is available (Maynard, et al., Interpharm Press, 1996; Dent, Urch Publ., 2001).

An effective amount of the compositions described herein may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of the compositions. Where there is more than one administration in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The disclosure is not limited to dosing intervals that are spaced equally in time, but encompass doses at non-equal intervals, such as a priming schedule consisting of administration at 1 day, 4 days, 7 days, and 25 days, just to provide a non-limiting example. In such aspects, various compositions can be administered using different dosing and spacing regiments. In such aspects, a first composition may be administered in one or more doses spaced at certain time intervals while a second composition may be administered in a different number of doses spaced at different time intervals. In such an aspect, a first composition and second composition may differ in makeup.

The compositions of the present invention can be administered in a dose, or dosages, where each dose comprises about 10 mg, 15 mg, 20 mg, 30 mg, 40 mg, 50 mg, 60 mg, 70 mg, 80 mg, 90 mg, 100 mg, 200 mg, 250 mg, 300 mg, 350 mg, 400 mg, 500 mg, 600 mg, and the like. The compositions of the present invention can be administered in a dose, or dosages, where each dose is dependent on subject body weight. As non-limiting examples, a dose, or dosages, can be administered at about 2 mg/kg, about 4 mg/kg, about 6 mg/kg, about 8 mg/kg, about 10 mg/kg, about 12 mg/kg, and the like. Various compositions disclosed herein can be administered at different dosages. As a non-limiting example, a first composition may be administered at one dosage while the second composition is administered at another composition.

A dosing schedule of, for example, once/week, twice/week, three times/week, four times/week, five times/week, six times/week, seven times/week, once every two weeks, once every three weeks, once every four weeks, once every five weeks, once every six weeks, and the like, is available for the compositions disclosed herein. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months. In some examples, similar benefit-risk profiles can be seen for two different dosing schedules. As a non-limiting example, a dosing schedule of 400 mg once every six weeks may have a similar benefit-risk profile in a subject undergoing a dosing schedule of 200 mg once every 3 weeks.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. Dosing schedules of the present disclosure can be related to cycles. A dosing schedule for a composition disclosed herein may be designed such that doses are given on certain days of a cycle. As a non-limiting example, doses may be administered on days 1, 15, and 29 of a repeating 42-day cycle. As a non-limiting example, cycle may repeat until a subject exhibits adverse side effects to doses. A cycle can additionally repeat until a subject is sufficiently cured of a disease. In this context, the term “about” means plus or minus one day, plus or minus two days, plus or minus three days, plus or minus four days, plus or minus five days, plus or minus six days, or plus or minus seven days.

Methods for co-administration with an additional therapeutic agent are well known in the art (Hardman, et al. (eds.) (2001) Goodman and Gilman's The Pharmacological Basis of Therapeutics, 10th ed., McGraw-Hill, New York, NY; Poole and Peterson (eds.) (2001) Pharmacotherapeutics for Advanced Practice: A Practical Approach, Lippincott, Williams & Wilkins, Phila., PA; Chabner and Longo (eds.) (2001) Cancer Chemotherapy and Biotherapy, Lippincott, Williams & Wilkins, Phila., PA).

An effective amount of a therapeutic agent is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

Administration of doses, or dosages, of compositions disclosed herein can be subject to change. The dosing and timing regimens may be changed according to factors known in the art. As non-limiting examples, administration of dosages of NC410 in combination with Pembrolizumab may be delayed allowing for resolution of any observed toxicities. Administration may resume if no medical condition or other circumstance exists that would make the participant unsuitable for further treatment.

In some examples, administration of compositions disclosed herein should not occur after certain times have elapsed. As a non-limiting example, an NC410 administration should generally not be delayed greater than 28-days between sequential doses, though this is subject to change. If a dose of NC410 or any other compositions disclosed herein is missed, a subject in need thereof may remain on the original treatment schedule with NC410 or any other compositions disclosed herein being administered at the time of next planned dose.

Formulations of therapeutic agents may be prepared for storage by mixing with physiologically acceptable carriers, excipients, or stabilizers in the form of, e.g., lyophilized powders, slurries, aqueous solutions, or suspensions.

Although several aspects have been disclosed in the foregoing specification, it is understood by those skilled in the art that many modifications and other aspects will come to mind to which this disclosure pertains, having the benefit of the teaching presented in the foregoing description and associated drawings. It is thus understood that the disclosure is not limited to the specific aspects disclosed hereinabove, and that many modifications and other aspects are intended to be included within the scope of any claims that can recite the disclosed subject matter.

### B. Formulations for Oral Administrations

In embodiments combination therapies and associated compositions are formulated for oral delivery. Oral delivery may include a singular solid or liquid dosage form described below comprising more than one composition (e.g., one composition of an immune checkpoint inhibitor and a second composition of an extracellular matrix binding component) or one or more solid or liquid dosage forms where compositions are separated. Dosage forms formulated for oral administration may be administered according to dosing and timing regimens described herein. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the disclosed. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the peptide (or chemically modified forms thereof) and inert ingredients which protect peptide in the stomach environment, and release of the biologically active material in the intestine.

The agents can be chemically modified so that oral delivery of the derivative is efficacious. Generally, the chemical modification contemplated is the attachment of at least one moiety to the component molecule itself, where the moiety permits uptake into the blood stream from the stomach or intestine, or uptake directly into the intestinal mucosa. Also desired is the increase in overall stability of the component or components and increase in circulation time in the body. PEGylation is an exemplary chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N. Y.) pp. 367-383; and Newmark, et al. (1982) J. Appl. Biochem. 4:185-189].

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. The agent can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e., the drug is enclosed in a semipermeable membrane which allows water to enter and push drug out through a single small opening due to osmotic effects.

For oral formulations, the location of release may be the stomach, the small intestine (the duodenum, the jejunem, or the ileum), or the large intestine. In some embodiments, the release will avoid the deleterious effects of the stomach environment, either by protection of the agent (or derivative) or by release of the agent (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™, cellulose acetate phthalate (CAP), Eudragit L™, Eudragit S™, and Shellac™. These coatings may be used as mixed films.

### C. Methods of Manufacture

### 1. Methods of Making Antibodies

Antibodies can be generated in cell culture, in phage, or in various animals, including but not limited to cows, rabbits, goats, mice, rats, hamsters, guinea pigs, sheep, dogs, cats, monkeys, chimpanzees, apes. Therefore, in one embodiment, an antibody is a mammalian antibody. Phage techniques can be used to isolate an initial antibody or to generate variants with altered specificity or avidity characteristics. Such techniques are routine and well known in the art. In one embodiment, the antibody is produced by recombinant means known in the art. For example, a recombinant antibody can be produced by transfecting a host cell with a vector comprising a DNA sequence encoding the antibody. One or more vectors can be used to transfect the DNA sequence expressing at least one VL and one VH region in the host cell. Exemplary descriptions of recombinant means of antibody generation and production include Delves, Antibody Production: Essential Techniques (Wiley, 1997); Shephard, et al., Monoclonal Antibodies (Oxford University Press, 2000); Goding, Monoclonal Antibodies: Principles And Practice (Academic Press, 1993); Current Protocols In Immunology (John Wiley & Sons, most recent edition).

The disclosed antibodies can be modified by recombinant means to increase greater efficacy of the antibody in mediating the desired function. Thus, it is within the scope of the invention that antibodies can be modified by substitutions using recombinant means. Typically, the substitutions will be conservative substitutions. For example, at least one amino acid in the constant region of the antibody can be replaced with a different residue. See, e.g., U.S. Pat. Nos. 5,624,821, 6,194,551, Application No. WO 9958572; and Angal, et al., Mol. Immunol. 30:105-08 (1993). The modification in amino acids includes deletions, additions, and substitutions of amino acids. In some cases, such changes are made to reduce undesired activities, e.g., complement-dependent cytotoxicity. Frequently, the antibodies are labeled by joining, either covalently or non-covalently, a substance which provides for a detectable signal. A wide variety of labels and conjugation techniques are known and are reported extensively in both the scientific and patent literature. These antibodies can be screened for binding to proteins, polypeptides, or fusion proteins of LAIR-1, LAIR-2, or Pembrolizumab. See, e.g., Antibody Engineering: A Practical Approach (Oxford University Press, 1996).

For example, suitable antibodies with the desired biologic activities can be identified using in vitro assays including but not limited to: proliferation, migration, adhesion, soft agar growth, angiogenesis, cell-cell communication, apoptosis, transport, signal transduction, and in vivo assays such as the inhibition of tumor growth. The antibodies provided herein can also be useful in diagnostic applications. As capture or non-neutralizing antibodies, they can be screened for the ability to bind to the specific antigen without inhibiting the receptor-binding or biological activity of the antigen. As neutralizing antibodies, the antibodies can be useful in competitive binding assays.

Antibodies that can be used in the disclosed compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

Also disclosed are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment.

Techniques can also be adapted for the production of single-chain antibodies specific to an antigenic peptide. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

A monoclonal antibody is obtained from a substantially homogeneous population of antibodies, i.e., the individual antibodies within the population are identical except for possible naturally occurring mutations that may be present in a small subset of the antibody molecules. Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

Monoclonal antibodies can be made using any procedure which produces monoclonal antibodies. In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

Antibodies may also be made by recombinant DNA methods. DNA encoding the disclosed antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques.

Methods of making antibodies using protein chemistry are also known in the art. One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, CA). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. Alternatively, the peptide or polypeptide is independently synthesized in vivo as described above. Once isolated, these independent peptides or polypeptides may be linked to form an antibody or antigen binding fragment thereof via similar peptide condensation reactions.

For example, enzymatic ligation of cloned or synthetic peptide segments allow relatively short peptide fragments to be joined to produce larger peptide fragments, polypeptides or whole protein domains. Alternatively, native chemical ligation of synthetic peptides can be utilized to synthetically construct large peptides or polypeptides from shorter peptide fragments. This method consists of a two-step chemical reaction. The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site.

### ii. Methods for Producing Proteins

The disclosed proteins, polypeptides, fragments, variants and fusions thereof can be manufactured using conventional techniques that are known in the art. Isolated fusion proteins can be obtained by, for example, chemical synthesis or by recombinant production in a host cell. To recombinantly produce a protein, polypeptide, fragment, variant or fusion thereof, a nucleic acid containing a nucleotide sequence encoding the protein, polypeptide, fragment, variant or fusion thereof can be used to transform, transduce, or transfect a bacterial or eukaryotic host cell (e.g., an insect, yeast, or mammalian cell). In general, nucleic acid constructs include a regulatory sequence operably linked to a nucleotide sequence encoding the protein, polypeptide, fragment, variant or fusion thereof. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well known in the art include, for example, Escherichia coli strains such as BL-21, and cultured mammalian cells such as CHO cells.

In eukaryotic host cells, a number of viral-based expression systems can be utilized to express fusion proteins. Viral based expression systems are well known in the art and include, but are not limited to, baculoviral, SV40, retroviral, or vaccinia based viral vectors.

Mammalian cell lines that stably express proteins, polypeptides, fragments, variants or fusions thereof, can be produced using expression vectors with appropriate control elements and a selectable marker. For example, the eukaryotic expression vectors pCR3.1 (Invitrogen Life Technologies) and p91023(B) (see Wong et al. (1985) Science 228:810-815) are suitable for expression of proteins, polypeptides, fragments, variants or fusions thereof, in, for example, Chinese hamster ovary (CHO) cells, COS-1 cells, human embryonic kidney 293 cells, NIH3T3 cells, BHK21 cells, MDCK cells, and human vascular endothelial cells (HUVEC). Additional suitable expression systems include the GS Gene Expression System™ available through Lonza Group Ltd.

Following introduction of an expression vector by electroporation, lipofection, calcium phosphate, or calcium chloride co-precipitation, DEAE dextran, or other suitable transfection method, stable cell lines can be selected (e.g., by metabolic selection, or antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, a protein, polypeptide, fragment, variant or fusion thereof, can be produced by (a) ligating amplified sequences into a mammalian expression vector such as pcDNA3 (Invitrogen Life Technologies), and (b) transcribing and translating in vitro using wheat germ extract or rabbit reticulocyte lysate.

Proteins, polypeptides, fragments, variants or fusions thereof, can be isolated using, for example, chromatographic methods such as affinity chromatography, ion exchange chromatography, hydrophobic interaction chromatography, DEAE ion exchange, gel filtration, and hydroxylapatite chromatography. In some embodiments, Proteins, polypeptides, fragments, variants or fusions thereof can be engineered to contain an additional domain containing amino acid sequence that allows the polypeptides to be captured onto an affinity matrix. For example, an Fc-fusion polypeptide in a cell culture supernatant or a cytoplasmic extract can be isolated using a protein A column. In addition, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify polypeptides. Fusion proteins can additionally be engineered to contain a secretory signal (if there is not a secretory signal already present) that causes the Proteins, polypeptides, fragments, variants or fusions thereof to be secreted by the cells in which it is produced. The secreted Proteins, polypeptides, fragments, variants or fusions thereof can then conveniently be isolated from the cell media.

### iii. Methods for Producing Isolated Nucleic Acid Molecules

Isolated nucleic acid molecules can be produced by standard techniques, including, without limitation, common molecular cloning and chemical nucleic acid synthesis techniques. For example, polymerase chain reaction (PCR) techniques can be used to obtain an isolated nucleic acid encoding a variant polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. Protein-encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992.

### D. Assays and Antibody Screening

Production of LAIR-2 Fc fusion protein (“LAIR-2-Fc”) for cancer therapy bypasses the need for development and screening of LAIR-1 mAbs. Because LAIR-2 has greater affinity than LAIR-1, in some embodiments, LAIR-2-Fc is selected as a therapeutic treatment over a LAIR-1 Fc fusion protein (“LAIR-1-Fc”). In some embodiments, LAIR-1-Fc may be utilized in mouse pre-clinical models because LAIR-2 does not exist in the mouse.

a. Assays for LAIR-2-Fc
1. Confirmation of the ability to bind multiple forms of collagen, SP-D, C1q and MBL by ELISA.
2. Confirmation of the ability of LAIR-2-Fc to inhibit binding of multiple collagens, SP-D and C1q to LAIR-1. This can be tested by: 1) ELISA competition assays, and 2) flow cytometry using LAIR-1 transfected cells incubated in the presence of titrated amounts of LAIR-2-Fc and fluorescently labeled LAIR ligands.
3. Analysis of binding affinity of LAIR-2-Fc to ligands in comparison to LAIR-1.
4. Functional assays to confirm LAIR-2-Fc prevents signaling by LAIR-1 expressing cells. Reporter cells may be utilized for these assays, or primary LAIR-1+ cells are another option.

### E. Method of Use

Evidence to date illustrates an inhibitory role for the LAIR-1 cell surface receptor and that LAIR-2 antagonized the function of LAIR-1 indirectly by binding identical ligands as LAIR-1, thus serving essentially as a decoy receptor. Tumor microenvironments are often rich in extracellular matrix proteins (ECMs), including the LAIR-1 ligand collagen (Rygiel et al., 2011, Mol. Immunol. 49:402-406). Therefore, LAIR-1 expressing cells localized to tumor microenvironments may be particularly suppressed through collagen cross-linking of LAIR-1 and subsequent inhibitory signaling. Increased LAIR-1 expression and signaling has been shown to inhibit the proliferation, differentiation, and function of several immune cell subsets, and thus is believed to suppress anti-tumor immunity, particularly in tumor microenvironments with high levels of the LAIR-1 ligands collagen, C1q and SP-D.

Both collagen and C1q have been shown to limit or alter antigen-presenting cell (monocyte/macrophage/dendritic cell (DC)) differentiation and activation through LAIR-1. LAIR-1 has also been found to be expressed on NK and T cells, but at much lower levels than on APCs. Nevertheless, studies have indicated that cross-linking LAIR-1 on NK cells and T cells can inhibit proliferation and function. Thus, it is believed that reducing LAIR-1 crosslinking can increase an immune response against cancer and infectious diseases. Increased levels of LAIR-2 are believed to promote anti-tumor immunity through the same mechanism. Therefore, soluble LAIR-1 and soluble LAIR-2 including LAIR-1 and LAIR-2 polypeptides and LAIR-1 and LAIR-2 fusion proteins, can be utilized for therapy of human diseases. For example, LAIR-2 Fc proteins can be used for cancer immunotherapy to enhance immune function by preventing ligand binding to LAIR-1. This strategy is particularly promising because LAIR-2 binds ligands with a higher affinity than LAIR-1.

Alternately, signaling through LAIR-1 on AML cancer cells that express high levels of LAIR-1 sustain the self-renewal capacity, or ‘stemness’, of AML cells by inhibiting apoptosis and differentiation through a unique LAIR-1-SHP-1-CAMK1-CREB pathway (Kang et al., 2015, Nat. Cell Biol. 17:665-677). In these cancers, decreased LAIR-1 signaling leads to AML cell death. Therefore, blockade (i.e., antagonism) of LAIR-1 signaling on leukemias is thought to be a treatment for the eradication of leukemias. As such, blockade of LAIR-1 signaling with LAIR-1 monoclonal antibodies, or with soluble LAIR-1 and soluble LAIR-2 including LAIR-1 and LAIR-2 polypeptides and LAIR-1 and LAIR-2 fusion proteins may be utilized for the treatment of leukemias by direct inhibition of cancer cell survival, as well as by promoting the anti-tumor immune response.

Conversely, decreased LAIR-1 expression or function is associated with several autoimmune manifestations, meanwhile, overexpression of LAIR-2 may promote autoimmunity through decoy binding of LAIR-1 ligands. LAIR-2 binding of LAIR-1 ligands can essentially reduce the cell surface cross-linking of LAIR-1, delimiting inhibitory signaling pathways leading to over-reactive immune function. Thus, it is believed that increasing LAIR-1 crosslinking can decrease an overactive or inappropriate immune response, for example in cases of autoimmune disease or inflammation. For example, blockade of LAIR-2 by mAbs could be utilized for treatment of autoimmune disease, as this would increase ligand binding to LAIR-1, thus downregulating immune responses. Targeting LAIR-2 would be particularly effective in diseases in which there is an imbalance between the expression of cell surface LAIR-1 and soluble LAIR-2, as has been shown for rheumatoid arthritis (Lebbink et al., 2008, J. Immunol 180:1662-1669).

Exemplary methods are discussed in more detail below.

### i. Therapeutic Strategies

Methods of inducing or enhancing an immune response in a subject are provided. Typically, the methods include administering a subject an effective amount of immunomodulatory agent, or cells primed ex vivo with the immunomodulatory agent. The immune response can be, for example, a primary immune response to an antigen or an increase effector cell function such as increasing antigen-specific proliferation of T cells, enhancing cytokine production by T cells, stimulating differentiation, or a combination thereof. In some embodiments, the agent can increase the development of naïve T cells into Th1, Th17, Th22, or other cells that secrete, or cause other cells to secrete, inflammatory molecules, including, but not limited to, IL-1β, TNF-α, TGF-beta, IFN-γ, IL-17, IL-6, IL-23, IL-22, IL-21, and MMPs. In some embodiments, the agent can reduce or inhibit the activity of Tregs, reduce the production of cytokines such as IL-10 from Tregs, reduce the differentiation of Tregs, reduce the number of Tregs, reduce the ratio of Tregs within an immune cell population, or reduce the survival of Tregs. The immunomodulatory agent can be administered to a subject in need thereof in an effective amount to overcome T cell exhaustion and/or T cell anergy. Overcoming T cell exhaustion or T cell anergy can be determined by measuring T cell function using known techniques.

The methods can be used in vivo or ex vivo as immune response-stimulating therapeutic applications. Thus, in some embodiments, the agent, or nucleic acid encoding the agent, is administered directly to the subject. In some embodiments, the agent or nucleic acid encoding the agent, is contacted with cells (e.g., immune cells) ex vivo, and the treat cells are administered to the subject (e.g., adoptive transfer). In general, the disclosed immunomodulatory agents can be used for treating a subject having or being predisposed to any disease or disorder to which the subject's immune system mounts an immune response. The agents can enable a more robust immune response to be possible. The disclosed compositions are useful to stimulate or enhance immune responses involving T cells.

The immunomodulatory agents utilized for increasing an immune response are typically those that reduce LAIR-1 expression, ligand binding, crosslinking, negative signaling, or a combination thereof. For example, the agent can be an antagonist of LAIR-1, such as an antagonist (blocking) anti-LAIR-1 antibody or antigen binding fragment thereof. In some embodiments, the antagonist binds to a LAIR-1 collagen binding domain (see, e.g., Brondijk, et al., Blood, 18(115):1364-73 (2010), and Zhou, et al., Blood, 127(5):529-537 (2016) and its supplemental information, which are specifically incorporated by reference in their entireties). In some embodiments, a LAIR-1 antagonist such as a function blocking antibody or functional fragment thereof specifically binds to an epitope including one or more of R59, E61, R62, E63, R65, S66, Y68, N69, 1102, R100, W109, E111, Q112, and Y115 of LAIR-1 (e.g., relative to SEQ ID NO:1). The agent can also be a LAIR-1 polypeptide, for example, a soluble polypeptide, or fusion protein thereof that can serve as a decoy receptor for one or more LAIR-1 ligands. The agent can also be LAIR-2 or a functional fragment or fusion protein thereof that can serve as a decoy receptor for one or more LAIR-1 ligands.

For example, in some embodiments an effective amount of a LAIR-2 fusion protein, for example LAIR-2-Fc, is administered to a subject with cancer or an infection. Treating patients with LAIR-2-Fc would result in decreased cross-linking of the LAIR-1 receptor, and subsequently, decreased inhibitory signaling of LAIR-1+ cells and improved immune function. Tipping the ratio towards increased levels of soluble LAIR-2 in comparison to cell surface LAIR-1, particularly in tumor microenvironments where the expression of LAIR-1/2 ligands are highly expressed, would favor enhanced anti-tumor immunity.

Tumor microenvironments with high levels of both collagens, SP-D and/or C1q, and with immune infiltrates that express high levels of LAIR-1 would be ideal for the disclosed immunotherapies, (e.g., LAIR-2-Fc immunotherapy). While ovarian cancers have high levels of collagen, it is unclear whether SP-D and C1q levels are high. Whereas, lung and GI cancers may have high levels of both collagens and SP-D, and therefore may be cancers to target with LAIR-2-Fc. In other embodiments, soluble LAIR-2, soluble LAIR-1, or a LAIR-1 fusion protein (e.g., LAIR-1-Fc) is utilized. In some embodiments, LAIR-2-based molecules may be selected because LAIR-2 binds ligands with a higher affinity than LAIR-1.

LAIR-1 blockade, for example using function blocking anti-LAIR-1 antibodies, can be an alternative agent or complementary agent to soluble LAIR-1 and LAIR-2 polypeptides and fusion proteins. For example, in some embodiments, LAIR-1 blockade and is combined with a decoy receptor such as soluble LAIR-1 or LAIR-2 or fusion protein thereof. The combined treatment (e.g., LAIR-2-Fe and LAIR-1 blockade) may be complementary.

In some embodiments, immune response stimulating therapy (e.g., in the treatment of cancer or infections) includes depletion of LAIR-1+ cells. LAIR-1 is highly expressed in mouse and human ovarian cancer ascites. The upregulation of LAIR-1 is restricted to immunoregulatory macrophages and F4/80+ DCs, both of which coexpress high levels of PD-L1. Therefore, targeting the depletion of LAIR-1 expressing cells would improve the overall condition of the tumor microenvironment by removal of immunoregulatory populations. While expression of LAIR-1 on other cell subsets in ovarian cancer have not been observed, because LAIR-1 is universally inhibitory, depletion of other LAIR-1+ cells would also have the effect of decreasing immune inhibition and improving anti-tumor immunity. LAIR-1 has also been shown to be expressed on the surface of, and is crucial for the development of acute myeloid leukemia cancers (Kang et al, Nature Cell Biology, Vol17, No 5, 2015; pp 665-679). Therefore, depletion of hematopoietic (‘blood’) cancers with LAIR-1 depleting mAbs would have the direct effect of reducing or eradicating LAIR-1 positive cancers.

### ii. Treatment of Cancer

The disclosed compositions and methods can be used to treat cancer. Generally, the agents are used to stimulate or enhance an immune response to cancer in the subject by administering to the subject an amount of an immunomodulatory agent that reduces LAIR-1 expression, ligand binding, crosslinking, negative signaling, or a combination thereof in combination with an immune checkpoint inhibitor (ICI). Administration includes injection, infusion, and other such known methods of administration according to dosing and timing regimens disclosed herein. The method can reduce one or more symptoms of the cancer.

The immune system is a proven defense against cancer initiation and growth. The regulation of immune responses is governed by cell surface interactions that direct immune cell function along specific pathways, including activation or inhibition against cancer cells. LAIR-1 is an inhibitory receptor on the surface of several immune cell (leukocyte) subsets that prevents optimal immune responses. Whereas, LAIR-2 is a soluble homolog that functions as a decoy to block LAIR-1 mediated inhibition. Pembrolizumab is an ICI that interacts with the PD-1 pathway.

In one embodiment, LAIR-2 Fc fusion protein promotes immune responses in vitro and in vivo. In another embodiment, LAIR-2 Fc reduces tumor growth and promotes survival. In still another embodiment, LAIR-2 Fc promotes anti-PD-1 immunotherapy in combination with an ICI (e.g., Pembrolizumab). The data provided herein demonstrate that LAIR-1 mAbs have in vitro activity in human T cell and myeloid cell lines, showing specific agonist and antagonist activity for specific mAb clones. These findings demonstrate the potential LAIR-1 pathway modulation by LAIR-2 Fe or LAIR-1 mAbs for immunotherapeutic intervention in cancer and other diseases.

In another embodiment, LAIR-2 Fc increases primary human T cell responsiveness to TCR stimulation. In another embodiment, LAIR-2 Fc increases antigen specific T cell responses in vivo.

Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless explicative potential, and sustained angiogenesis. The term “cancer cell” is meant to encompass both pre-malignant and malignant cancer cells. In some embodiments, cancer refers to a benign tumor, which has remained localized. In other embodiments, cancer refers to a malignant tumor, which has invaded and destroyed neighboring body structures and spread to distant sites. In yet other embodiments, the cancer is associated with a specific cancer antigen (e.g., pan-carcinoma antigen (KS 1/4), ovarian carcinoma antigen (CA125), prostate specific antigen (PSA), carcinoembryonic antigen (CEA), CD19, CD20, HER2/neu, etc.).

The methods and compositions disclosed herein are useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xenoderma pegmentosum, keratoactanthoma, seminoma, thyroid follicular cancer and teratocarcinoma.

Cancers caused by aberrations in apoptosis can also be treated by the disclosed methods and compositions. Such cancers may include, but are notlimited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions in the ovary, bladder, breast, colon, lung, skin, pancreas, or uterus. In other specific embodiments, sarcoma, melanoma, or leukemia is treated or prevented by the methods and compositions.

The disclosed compositions and methods are particularly useful for the treatment of cancers that are associated with cells that express abnormally high levels of LAIR-1, high levels of LAIR-1 ligand, low levels of LAIR-2, or a combination thereof.

Specific cancers and related disorders that can be treated or prevented by methods and compositions disclosed herein include, but are not limited to, leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as, but not limited to, Hodgkin's disease or non-Hodgkin's disease lymphomas (e.g., diffuse anaplastic lymphoma kinase (ALK) negative, large B-cell lymphoma (DLBCL); diffuse anaplastic lymphoma kinase (ALK) positive, ALK+ anaplastic large-cell lymphoma (ALCL), acute myeloid lymphoma (AML)); multiple myelomas such as, but not limited to, smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including, but not limited to, ocular melanoma such as iris melanoma, choroidal melanoma, and cilliary body melanoma, and retinoblastoma; vaginal cancers, including, but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, carcinosarcoma. In addition, cancers include myxosarcoma, osteogenic sarcoma, endotbeliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

It should be emphasized that the above-described aspects are merely possible examples of implementations, merely set forth for a clear understanding of the principles of the present disclosure. Any process descriptions or blocks in flow diagrams should be understood as representing modules, segments, or portions of code which comprise one or more executable instructions for implementing specific logical functions or steps in the process, and alternate implementations are included in which functions may not be included or executed at all, can be executed out of order from that shown or discussed, including substantially concurrently or in reverse order, depending on the functionality involved, as would be understood by those reasonably skilled in the art of the present disclosure. Many variations and modifications can be made to the above-described aspect(s) without departing substantially from the spirit and principles of the present disclosure. Further, the scope of the present disclosure is intended to cover any and all combinations and sub-combinations of all elements, features, and aspects discussed above. All such modifications and variations are intended to be included herein within the scope of the present disclosure, and all possible claims to individual aspects or combinations of elements or steps are intended to be supported by the present disclosure.

### EXAMPLES

### Example 1. Safety and Tolerability Study of NC410 in Combination with Pembrolizumab

### Methods:

Safety and tolerability will be assessed by monitoring frequency, duration, and severity of adverse events (AEs). Toxicity grading per NCI CTCAE v5.0. The study will be split into two phases including Phase 1b and Phase 2, as described below. The Phase 1b portion of the study will enroll participants with advanced unresectable and/or metastatic solid tumors including CRC, Gastric including GE junction, Esophageal, Ovarian, and H&N cancers regardless of prior treatment with immune checkpoint inhibitors (ICIs) or microsatellite stable/microsatellite instable (MSS/MSI) status, as shown in FIG. 3. Participants include men and women, 18 years or older. Participants must provide written informed consent and have adequate organ function.

Participants must present with measurable disease based on RECIST v1.1 and consent to have a non-target lesion biopsied prior to and during treatment. RECIST v1.1 will be adapted to account for the unique tumor response characteristics seen with immunotherapy (Chiou, V. L., et al., “Pseudoprogression and Immune-Related Response in Solid Tumors”, J Clin Oncol., 33:3541-3 (2015)). Immunotherapeutic agents may produce antitumor effects by potentiating endogenous cancer-specific immune responses. The response patterns seen with such an approach may extend beyond the typical time course of responses seen with cytotoxic agents and can manifest a clinical response after an initial increase in tumor burden or even the appearance of new lesions. Standard RECIST may not provide an accurate response assessment of immunotherapeutic agents. Immunotherapy RECIST (IRECIST) is RECIST 1.1 adapted as described below to account for the unique tumor response seen with immunotherapeutics. Following site identification of progressive disease, iRECIST will be used by site investigators to assess tumor response progression and make treatment decisions. Archival tissue may be submitted in place of fresh biopsy at pre-treatment. On-treatment biopsy will be fresh biopsy. If a participant is scheduled to have a tumor biopsy for the purposes of this study and it is subsequently determined that tumor tissue cannot safely be obtained, then the participant may still enroll in the study. Participants with certain serious medical conditions (in addition to the diagnosis of cancer) would be excluded from participation in the trial.

The recommended Phase 2 dose (RP2D) of NC410 (LAIR-2-Fc) when combined with a standard dose of Pembrolizumab will be defined in participants with advanced unresectable and/or metastatic ICI refractory solid tumors (regardless of MSI status), or ICI naïve MSS/MSI-low solid tumors during the safety and tolerability study of Phase 1b.

All eligible participants, after signing an Informed Consent Form (ICF), will be allocated by non-random assignment, and will receive a unique participant number with the first 4 digits serving as the site number. The participant numbering will be managed by the sites. Investigative sites must complete all applicable CRFs for participants consented to the trial, even if the participant is not treated with study drug (i.e., screen failure). Dose level and cohort assignment will occur at time of participant enrollment and as indicated by the sponsor or designee. Dose level and cohort assignment will be maintained within the CRF per the CRF completion guidelines and managed by the Sponsor/CRO.

Participants will receive Pembrolizumab on Day 1 of each 42-day cycle followed by administration of NC410. Additional doses of NC410 will be given on Day 15 and Day 29 or on a weekly basis of each 42-day cycle as shown in FIG. 4 and described in Table 1 below.

TABLE 1
Trial Treatment

| Intervention | Dosage | Unit Dose | Dosage | Route of | |
|---|---|---|---|---|---|
| Name | Formulation | Strength(s) | Level(s) | Administration | Regimen |
| KEYTRUDA © | Solution for | 100 mg/vial | 400 mg | IV infusion | Day 1 of each |
| (Pembrolizumab) | infusion | | Q6W | | 42-day cycle |
| NC410 | Solution for | 16 mg/vial or | RP2D | IV infusion | Day 1, Day |
| | infusion | 320 mg/vial* | Q2W | | Day 15 and |
| | | *depending on | | | Day 29 or on |
| | | dose level | | | a weekly |
| | | | | | basis of each |
| | | | | | 42-day cycle |

Abbreviations: IV = intravenous; Q2W = every 2 weeks; Q6W = every 6 weeks; RP2D = recommended Phase 2 Dose.
*Depending on dose level.

The study will be conducted in two phases. Phase 1b will include dose escalation of NC410 to determine the optimal dose administration schedule and RP2D when combined with a standard dose of Pembrolizumab. Phase 2 will include dose expansion to evaluate the recommended Phase 2 dose (RP2D) of NC410 in combination with a standard dose of Pembrolizumab. Participants will continue until progressive disease, unacceptable adverse events (AEs), intercurrent illness that prevents further administration of treatment, an investigator's decision to withdraw the participant, a participant withdraws consent, pregnancy of the participant, noncompliance with trial treatment or procedure requirements, participant receives 18 treatments (approximately 2 years) of Pembrolizumab, or administrative reasons requiring cessation of treatment occur.

Participants who discontinue for reasons other than progressive disease will have post-treatment follow-up for disease status until progressive disease, initiating a non-study cancer treatment, withdrawing consent, or becoming lost to follow-up. All participants will be followed for overall survival (OS) until death, withdrawal of consent, or the end of the study. After the end of treatment, each participant will be followed for 30 days for AE monitoring. Serious adverse events (SAE) and events of clinical interest (ECI) will be collected for 90 days after the end of treatment or for 30 days after the end of treatment if the participant initiates new anticancer therapy, whichever is earlier.

In Phase 1b, a modified Toxicity Probability Interval (mTPI) (Ji, Y., et al., “Modified toxicity probability interval design: a safer and more reliable method than the 3+3 design for practical phase I trials”, J Clin Oncol., 31:1785-91 (2013)) with a target dose limiting toxicity (DLT) rate of approximately 30% will be applied for dose escalation and confirmation to determine a recommended Phase 2 dose (RP2D) for NC410 in combination with Pembrolizumab.

There will be five dose levels of NC410 evaluated during the Phase 1b portion of the study. NC410 is in frozen liquid form formulated for iv infusion. The drug product is provided in a 1 mg/mL (16 mg/vial) and 20 mg/mL (320 mg/vial) concentration.

The pre-determined dose levels of NC410 (in combination with a fixed dose of Pembrolizumab of 400 mg) will be explored independently. The dose levels are as follows:
Dose Level-1 (de-escalating dose): 15 mg
Dose Level 1 (starting dose): 30 mg
Dose Level 2:60 mg
Dose Level 3:100 mg
Dose Level 4:200 mg

The starting dose will be 30 mg (Dose Level 1) of NC410. A de-escalation dose of NC410 is available if the starting dose of NC410 is deemed not tolerable in combination with Pembrolizumab. All dose escalation and de-escalation decisions will be based on the occurrence of dose limiting toxicities (DLTs) at a given dose during the first 42-day period (i.e., Cycle 1) also referred to as the DLT observation period and will be made jointly by the investigators and the sponsor. All DLTs will be assessed by the investigator using National Cancer Institute Common Terminology Criteria for Adverse Events (NCI CTCAE) v5.0. The dose of Pembrolizumab will remain constant at 400 mg administered every 6 weeks for each dose level for NC410 and in each cohort through Phase 2.

Pembrolizumab 400 mg will be administered by intravenous (iv) infusion, over a minimum of 30 minutes on Day 1 of each 42-day cycle followed by NC410 iv infusion over a minimum of 30 minutes, with 30-60 minutes between administration of each study treatment. However, given the variability of infusion pumps from site to site, a window between −5 minutes and +10 minutes is permitted (i.e., infusion time is 30 minutes (−5 min/+10 min).

NC410 will also be administered on Day 1, Day 15 and Day 29 or on a weekly basis of each 42-day cycle at 15 mg, 30 mg, 60 mg, 100 mg or 200 mg, as a stand-alone solution or in combination with Pembrolizumab. NC410 may also, be administered on a weekly basis at 100 mg, as a stand-alone solution or in combination with Pembrolizumab. Participants will continue until progressive disease, unacceptable AEs, intercurrent illness that prevents further administration of treatment, investigator's decision to withdraw the participant, participant withdraws consent, pregnancy of the participant, noncompliance with trial treatment or procedure requirements, participant receives 18 treatments (approximately 2 years) of Pembrolizumab, or administrative reasons requiring cessation of treatment.

In FIG. 5, the number of participants treated is indicated in the columns and the number of participants who experienced a DLT is indicated in the rows. Dosing decisions shown in FIG. 5 include: escalate to the next higher dose (E), stay at the current dose(S), de-escalate to the next lower dose (D), and de-escalate to a lower dose and never test this dose again (i.e., unacceptably toxic dose; DU). Alternative dose levels or administration schedules may be considered pending safety review and emerging data.

As a non-limiting example, the dose escalation rules can proceed as follows if 3 participants are enrolled: if 0 out of the first 3 participants at a given dose level develops a DLT, then the dose can be escalated with the next available participant enrolled to the next level cohort without further expansion. If 1 out of the first 3 participants at a given dose level develops a DLT, no more than an additional 3 participants should be enrolled at this dose level until additional DLT data are available. This dose would be considered unacceptably toxic if all 3 of the additional participants experience a DLT (i.e., 4 out of 6 participants). If 2 out of the first 3 participants at a given dose level develop a DLT, the dose will be de-escalated to the lower dose level cohort. If 3 out of the first 3 participants at a given dose level develop a DLT, this dose will be considered unacceptably toxic (i.e., the dose will be de-escalated and never re-escalated to that dose again). The same principle will be applied whether 3, 4, 5 or 6 participants are enrolled in the same dose cohort according to FIG. 5.

Based on the mTPI design, the number of participants who are enrolled at a dose, but are not yet fully evaluable for DLT assessment, may not exceed the number of remaining participants who are at risk of developing a DLT before the dose would be considered unacceptably toxic (denoted as DU in FIG. 5). To determine how many more participants can be enrolled at a dose level, one can count steps in a diagonal direction (down and to the right) from the current cell to the first cell marked DU. In total, 3 to 14 participants may be enrolled at a given dose level.

Dose escalation and confirmation will end after 10 evaluable participants have been treated at any of the selected doses. The pool-adjacent-violators-algorithm (Ji, Y., et al., “Modified toxicity probability interval design: a safer and more reliable method than the 3+3 design for practical phase I trials”, J Clin Oncol., 31:1785-91 (2013)) will be used to estimate the DLT rates across doses.

The dose with an estimated DLT rate closest to 30% may be treated as a preliminary maximum tolerated dose (MTD). The totality of the data will be considered before a dose is selected to carry forward to Phase 2 and the escalation schedule may be adjusted based on pharmacokinetic (PK), pharmacodynamics (PD), and safety data emerging throughout the study to determine the RP2D.

If a participant is not evaluable for the DLT observation period for any reason, they may be replaced with the next available participant if escalation or de-escalation rules have not been fulfilled. The dose level cohort which is determined to be the RP2D will be expanded until at least 10 participants have been dosed and observed for the duration of the 42-day DLT Observation period before moving onto Phase 2 Dose Expansion.

### Example 2. Clinical Study of NC410 in Combination with Pembrolizumab

### Methods:

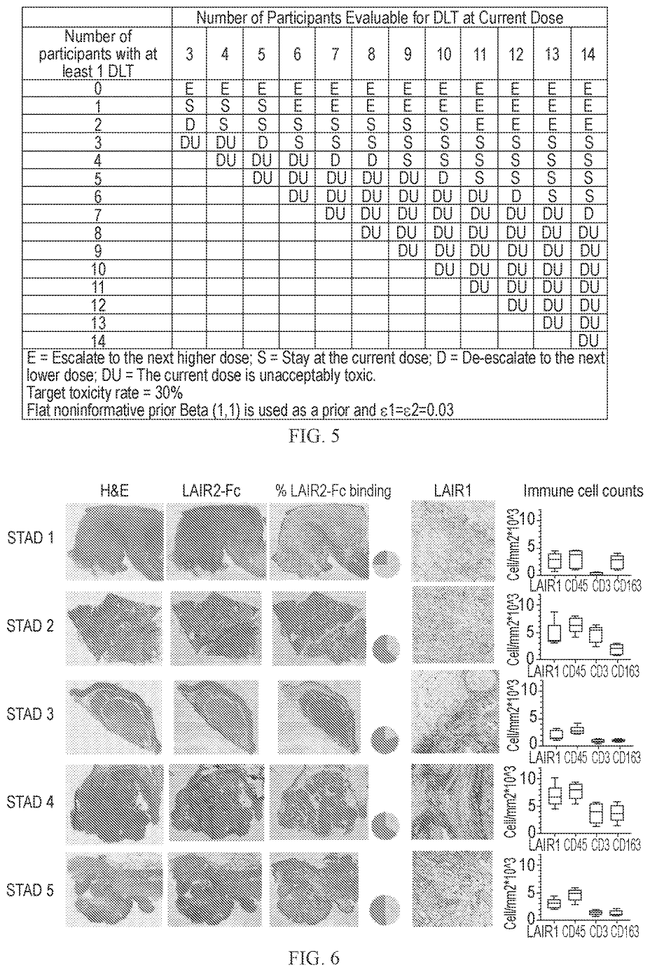

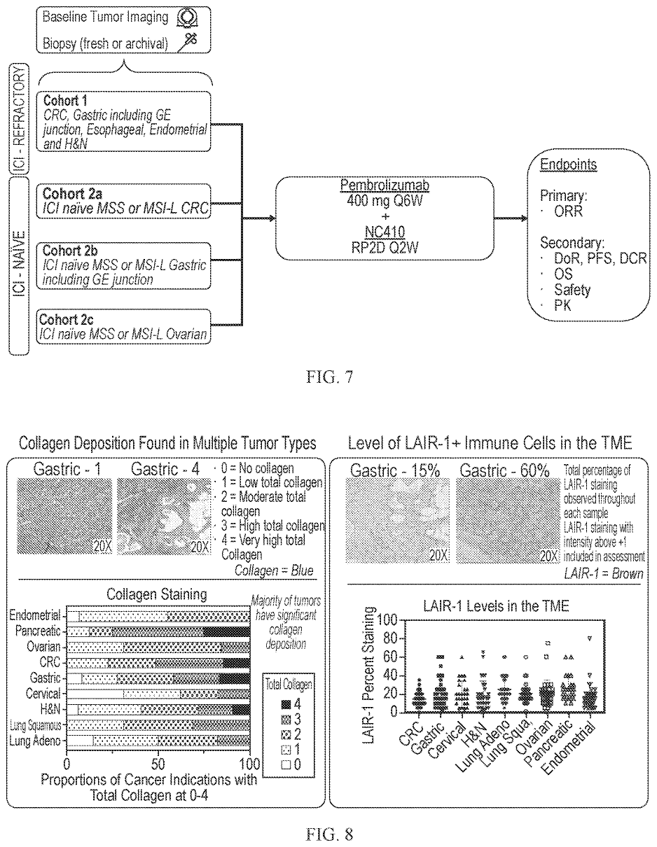

Selection parameters for study participants, as well as parameters applicable to withdrawing participants from a study, used in Phase 1b above (i.e., safety and tolerability study) are also applicable to Phase 2 described below (i.e., clinical study). Immunohistochemical (IHC) studies were conducted to further inform identification of potential tumor types for NC410 therapy. These IHC studies were also based on criteria from earlier in-depth nonclinical analyses of tumor types. Selection of tumor types for IHC studies utilized the following criteria: LAIR-1 expression in the TME, CD163 M2-like macrophage marker, as assessed for relative increase in cancer vs normal tissues, and analysis of several LAIR ligands, as assessed for overexpression in the TME in comparison to normal tissues. To assess these markers, IHC analysis, including hematoxylin and eosin (H&E) and trichrome staining, LAIR-1 expression, LAIR-2-Fc (NC410) binding and immune cell infiltration, was performed. Example of these results from the analysis of the stomach adenocarcinoma (STAD) are shown in FIG. 6. For LAIR-2-Fc binding, brown staining indicates the binding of LAIR-2 Fe, and blue staining is hematoxylin counterstaining indicating no binding by LAIR-2 Fc. LAIR-2 Fe positive (green) and negative areas (red) were quantified as demonstrated in the associated pie charts. Five regions of interest (ROI) with a diameter of 600 μm were randomly chosen in the LAIR-2 Fe positive area for immune cell quantification. One of the ROIs was magnified to show the LAIR-1 staining. The graph shows the quantification of immune cells. The cell counts of LAIR-1+, CD45+, CD3+ and CD163+ cells within the five ROI were quantified and calculated as ×103/mm2.

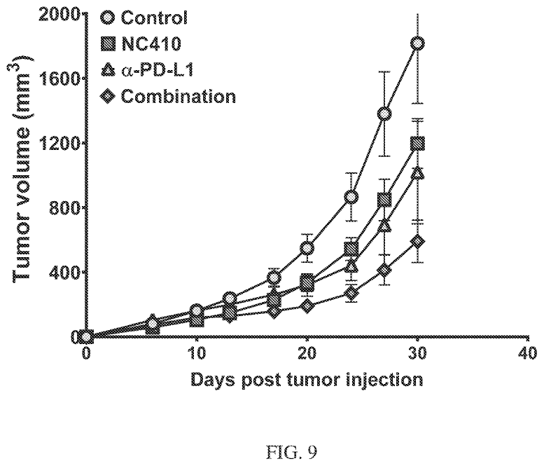

Phase 2 of the study will further evaluate the clinical benefit of the recommended phase 2 dose (RP2D) of NC410 obtained in Phase 1b in combination with standard dose Pembrolizumab in participants with advanced unresectable and/or metastatic solid tumors across different cohorts as outlined below based on previous immune checkpoint inhibitor (ICI) refractory or naïve history (as shown in FIG. 7). Assessment of antitumor activity will be used to evaluate the clinical benefit of NC410 in combination with Pembrolizumab and confirm pre-clinical studies, showing NC410 in combination with anti-PD-L1 results in synergistic and reproducible tumor killing in murine models (FIG. 9).

Factors including but not limited to Objective Response Rate (ORR), Disease Control Rate (DCR), Duration of Response (DoR), Progression-free Survival (PFS) (based on RECIST v1.1 as assessed by the investigator), and Overall Survival (OS) will be assessed. Assessment of pharmacokinetics (PK) of NC410 concentration in serum, as well as assessment of combination treatment on the pharmacokinetic/pharmacodynamics (PK/PD) profile will be observed.

The study will enroll ICI Refractory solid tumors in Cohort 1 and ICI Naïve solid tumors in Cohorts 2a, 2b, and 2c, as described below. Microsatellite stable (MSS) or microsatellite instability-low (MSI-L) status must be confirmed prior to entry into these cohorts (either by historical result or during screening). Cohorts are defined as the following:

Cohort 1: ICI Refractory Solid Tumors (Colorectal Cancer Microsatellite Instability-High (CRC MSI-H), Gastric cancer including Gastroesophageal (GE) junction, Esophageal cancer, Endometrial cancer, and head and neck (H&N) cancer).

Cohort 2a: ICI Naïve MSS or MSI-L CRC

Cohort 2b: ICI Naïve MSS or MSI-L Gastric including GE junction

Cohort 2c: ICI Naïve MSS or MSI-L Ovarian

### RU2021129474A

The invention relates to an antibody or antigen-binding fragment thereof specifically binding to LAIR-1, wherein the antibody or antigen-binding fragment thereof contains light chain complementarity determining regions having the amino acid sequence shown in SEQ ID NO: 100, SEQ ID NO: 101 and SEQ ID NO: 102 and complementarity-determining heavy chain regions having the amino acid sequence shown in SEQ ID NO: 104, SEQ ID NO: 105, and SEQ ID NO: 106. It further relates to a method of treating leukemia in a subject in need, comprising administering to the subject an effective amount of a pharmaceutical composition comprising an anti-LAIR-1 monoclonal antibody, a soluble LAIR-1 polypeptide, a soluble LAIR-2 polypeptide, an LAIR-1 fusion protein, an LAIR-2 fusion protein or combinations thereof, to inhibit or reduce LAIR-1 signaling in leukemia cells, so as to inhibit the survival of leukemia cells or promote an antitumor immune response against leukemia cells.

1. An antibody or antigen-binding fragment thereof specifically binding to LAIR-1, wherein the antibody or antigen-binding fragment thereof contains light chain complementarity determining regions having the amino acid sequence shown in SEQ ID NO: 100, SEQ ID NO: 101 and SEQ ID NO: 102 and complementarity-determining heavy chain regions having the amino acid sequence shown in SEQ ID NO: 104, SEQ ID NO: 105, and SEQ ID NO: 106.

2. An antibody according to claim 1, containing a light chain variable region according to SEQ ID NO: 99 and a heavy chain variable region according to SEQ ID NO: 103.

3. An antibody according to claim 1 or 2, containing a heavy chain with the amino acid sequence shown in SEQ ID NO: 138 or SEQ ID NO: 140, and a light chain with the amino acid sequence shown in SEQ ID NO: 142.

4. A nucleic acid sequence encoding an antibody according to any one of paragraphs. 1-3.

5. A cell that constitutively or inducibly expresses an antibody or antigen-binding fragment thereof that specifically binds to LAIR-1, where the cell contains the nucleic acid according to claim 4.

6. Pharmaceutical composition containing an effective amount of an antibody according to any one of paragraphs. 1-4, wherein an effective amount of the composition enhances an immune response in a subject in need thereof.

7. The pharmaceutical composition of claim 6 wherein the immune response is a primary immune response to an antigen or an increase in effector cell function such as an increase in antigen-specific T cell proliferation, an increase in T cell cytokine production, stimulation of differentiation, or a combination thereof.

8. A method for enhancing an immune response in a subject in need thereof, comprising administering to the subject a pharmaceutical composition according to claim 6 or 7.

9. The method of claim 8 wherein the subject has inflammation.

10. The method of claim 8, wherein the subject has an autoimmune disorder.

11. The method of claim 10 wherein the autoimmune disorder is rheumatoid arthritis.

12. The method according to any one of paragraphs. 8-11, wherein the subject or disease or condition is characterized by decreased LAIR-1 expression, decreased LAIR-1 ligand expression, increased LAIR-2 expression, or a combination thereof.

13. The method according to any one of paragraphs. 8-12, further comprising administering a second therapeutic agent to the subject.

14. The method of claim 12 or 13, wherein LAIR-1 is expressed on a myeloid cell, a T cell, a natural killer (NK), or a combination thereof.

15. The method of claim 14 wherein the myeloid cell is an antigen presenting cell.

16. The method of claim 15 wherein the antigen presenting cell is a monocyte, macrophage, or dendritic cell.

17. A method of treating leukemia in a subject in need, comprising administering to the subject an effective amount of a pharmaceutical composition comprising an anti-LAIR-1 monoclonal antibody, a soluble LAIR-1 polypeptide, a soluble LAIR-2 polypeptide, an LAIR-1 fusion protein, an LAIR-2 fusion protein or combinations thereof, to inhibit or reduce LAIR-1 signaling in leukemia cells, so as to inhibit the survival of leukemia cells or promote an antitumor immune response against leukemia cells.

18. The method of claim 17 wherein the leukemia is acute myeloid leukemia.
